SEQUENCE LISTING (29 sequences)

SEQ ID NO: 1 - protein, 296 aa, Talaromyces leycettanus
MHRPLQLWALAALTSLVTAAPAPVLRRDVSSSVLSELDLFAQYSAAAYCSSNIGSPGTKLTCSVGNCPRVEAADTETLIEFNESSSFGDVTGYIAVDRTNSLLVLAFRGSSTVSNWEADLDFPLTDASSLCSGCEIHSGFWAAWQTVQASITSTLESAIASYPGYTLVFTGHSYGAALAAIAATTLRNAGYTIQLYDYGQPRLGNLALAQYITAQTQGANYRVTHTDDIVPKLPPELFGYHHFSPEYWITSGDNVTVTTSDVQVVTGIDSTAGNDGTLLDSTSAHDWYIVYIDGCD

SEQ ID NO: 2 - protein, 269 aa, Talaromyces leycettanus
DVSSSVLSELDLFAQYSAAAYCSSNIGSPGTKLTCSVGNCPRVEAADTETLIEFNESSSFGDVTGYIAVDRTNSLLVLAFRGSSTVSNWEADLDFPLTDASSLCSGCEIHSGFWAAWQTVQASITSTLESAIASYPGYTLVFTGHSYGAALAAIAATTLRNAGYTIQLYDYGQPRLGNLALAQYITAQTQGANYRVTHTDDIVPKLPPELFGYHHFSPEYWITSGDNVTVTTSDVQVVTGIDSTAGNDGTLLDSTSAHDWYIVYIDGCD

SEQ ID NO: 3 - DNA, 1457 bp, Cryphonectria parasitica
  source 1..1457: mol_type = genomic DNA
cagttgagtccggatgtgtttggccttcatccgctccctttctgcacacggtctttagcccagccagcctgccagcctgctagccagccagacagacaagatgagactgtcggtcgcctcctcactttccctggcctctggcctggtggcagcctcgcctcttcaggctcgtggtaagtagagcttggactcaacctctccgttgaaggctatcttgaacctaattgtgattatacacagactccgccgtcctggacgagacgagttacgaaaacatgaagtactacgtgcagtatgctgcgtctgcgtactgcgacagcgaagatgctgtgggcacactcgtatcctgcggtgactctgggtgtcctaatgtcacggcgaatggtgccaccatcgtcggtaccatgccgtgagtctttccttagcccccgaggactttatgctccttgacatcagagagcccagatgctaatatttgcgactccagcaccaccaccaccttcgacctggaaggctatgtggccgtcgaccccacccgcgaagaaattgtggtcgcattccgtggctcatctgacctgcgcaactggattgcagacttcgacttcattgaggttgcgtactcggattgcactggctgctatgtccacgatggattctacgaatcctggaaggaaattcagacctatgccgtcgggtacgtcgaggcggcgtacgagacgtacccagactataccctcgtcatcacaggtcactccctgggcgccgccgtcgccaccctcgcgggtgtgcagttcagaatcgacgggtgagtaaaaccaacacacaaaaggactcatgtgagacgtccacatagtaccgtgctgacggaactattcaaagctacccttgtgatatctacactgtcggctccccccgcatcggcaacctggcatgggccgagtttgtgaccgcccaggatggcgctgaataccgggctacccattacgatgaccccgtgccgcgcctgccacccatcgtgcttggctactaccacacgagccccgagttctggctcgccgccggtcccgccaccaacatcgactacaccattgacggaattgatgtctgcgtaggcaacgcaaacaccagttgcaacgccggcacgactggctttgacgccgatgcgcacgaatactacttccagtacatgggctgtggcgatgagagcaacggcattgacatgcgcaagcggtcccccaatactctgtttggtcgtgacggcagcagcaacatcaccgatgcggagctggcccagaagctcaccaactacacgatgcaagatatcgccttttcggagaagctcgcctccagcagctaagcctggtaatgctcggatcagaggcgcggtcggtatgaggacaattccatgaggtcactggtaggggcgatcgttcccgtccaacatcaaaagtggtggg

SEQ ID NO: 4 - protein, 333 aa, Cryphonectria parasitica
MRLSVASSLSLASGLVAASPLQARDSAVLDETSYENMKYYVQYAASAYCDSEDAVGTLVSCGDSGCPNVTANGATIVGTMPTTTTFDLEGYVAVDPTREEIVVAFRGSSDLRNWIADFDFIEVAYSDCTGCYVHDGFYESWKEIQTYAVGYVEAAYETYPDYTLVITGHSLGAAVATLAGVQFRIDGYPCDIYTVGSPRIGNLAWAEFVTAQDGAEYRATHYDDPVPRLPPIVLGYYHTSPEFWLAAGPATNIDYTIDGIDVCVGNANTSCNAGTTGFDADAHEYYFQYMGCGDESNGIDMRKRNITDAELAQKLTNYTMQDIAFSEKLASSS

SEQ ID NO: 5 - protein, 315 aa, Cryphonectria parasitica
SPLQARDSAVLDETSYENMKYYVQYAASAYCDSEDAVGTLVSCGDSGCPNVTANGATIVGTMPTTTTFDLEGYVAVDPTREEIVVAFRGSSDLRNWIADFDFIEVAYSDCTGCYVHDGFYESWKEIQTYAVGYVEAAYETYPDYTLVITGHSLGAAVATLAGVQFRIDGYPCDIYTVGSPRIGNLAWAEFVTAQDGAEYRATHYDDPVPRLPPIVLGYYHTSPEFWLAAGPATNIDYTIDGIDVCVGNANTSCNAGTTGFDADAHEYYFQYMGCGDESNGIDMRKRNITDAELAQKLTNYTMQDIAFSEKLASSS

SEQ ID NO: 6 - DNA, 1824 bp, Gloeophyllum trabeum
  source 1..1824: mol_type = genomic DNA
cattccgtgtcggcatttataccagcagggtgtatagaggggccatgcgagccagccctacaacattcagcgcaacccgctcgaactccaagctctgcagatggcagcttcgttcgctttagcagttgcgctcttggttggtgctgtcgtggatgctgcccccaatctacagaagcgtcagtctataacgacgttgtcttcctcgcaagtctcagctttcacgccttacacatggtatgcgagcgccgggtattgctcaccttccacaaccttgacatggacctgcggaagtaagtcgggttgtgtttcccatcggaaagtagcactgactgtgtcaactcagccaattgccaagctaacagtgggttcgaacccgtggcttctggaggcgatggtgattccactcagtactgtaagcttagtaccgtacaccttgcgcttagtcagattaatgagcgacttcctaaagggtttgttgggtatgacccgtctctggacaccgtgatcgtctcgcatcaaggcacggacacttcggagatgtaggtgtcgcctacttaatcgcacctagaaacatacctagctaaccctggtgttccctggtagcctccctctgctgacagacgccgatatcgagaagacgactcttgattcgtcactattcccgggtataagctcctctattgaggtgcacgctgggttcgcagatgcgcaagccaacgccgccacggacgtcttagcggctgtccagacagcactgtcgaagtatagcacgaacaccgttactatggtcggacactcccttggtgcgttgcacatctgcccacatttctgaattgacgctcacctgctgtgaaggtgccgcaatttctctgttggatctggtttaccttcctttgcacttgccctccgggctcacttacaaaatgtacggttacggaatgcccagggtatgtctacttcctttgggaccaggggtgacaagttgaccaggacttctaggttggcaaccaggccttcgctgactacatcgacgcgaaccatcccatgactcacgtgaacaataaggaagactacatccccatcctacccgggatgttcttaggcttcgttcatccctctggcgaggtccacatcgaagactcgggatcatgggatgcctgcccaggtacgtctgcccaggggacttatccatttcattggctcatttcagaccgcaggtcaagacaaccccagcactctatgcatcgtcggcgatgtcccggagatttgggatggtgacgaatctgatcatgacggaccctacgggccagtcactatgggttgctaaaataacctgtactttcttttgcggtcggactggtgccaaatcaaaactgtactatgtcttcactgttcattggtacggctgcacgggccgaggtcgtgcgagtggacttcccatgttcattgttggcacgcgaacgcgaaggtgctctgacaggaagacatcacccggagggcgttcaagatgagaaggtagggaaggtcaggtgcttgtggtcgtttgcgatctctcagcgattgatcgtcatactcttccacatacccatatctcttatcacccatgaaagaccgtaggattgacgctagccgcacgcctccgtagcggtcctcgaagcaggcccaccaaatctgaacaacccgaaaatcatggtccccgggcaatttggtgggacattcgttgaccccaaggcaagcttctccggaatagcctatgtcatccctgctgagaacaatcctgttgtagtatgactggtgcttctcgacagtctcgctgaaggattg

SEQ ID NO: 7 - protein, 298 aa, Gloeophyllum trabeum
MAASFALAVALLVGAVVDAAPNLQKRQSITTLSSSQVSAFTPYTWYASAGYCSPSTTLTWTCGTNCQANSGFEPVASGGDGDSTQYWFVGYDPSLDTVIVSHQGTDTSEILPLLTDADIEKTTLDSSLFPGISSSIEVHAGFADAQANAATDVLAAVQTALSKYSTNTVTMVGHSLGAAISLLDLVYLPLHLPSGLTYKMYGYGMPRVGNQAFADYIDANHPMTHVNNKEDYIPILPGMFLGFVHPSGEVHIEDSGSWDACPGQDNPSTLCIVGDVPEIWDGDESDHDGPYGPVTMGC

SEQ ID NO: 8 - protein, 279 aa, Gloeophyllum trabeum
APNLQKRQSITTLSSSQVSAFTPYTWYASAGYCSPSTTLTWTCGTNCQANSGFEPVASGGDGDSTQYWFVGYDPSLDTVIVSHQGTDTSEILPLLTDADIEKTTLDSSLFPGISSSIEVHAGFADAQANAATDVLAAVQTALSKYSTNTVTMVGHSLGAAISLLDLVYLPLHLPSGLTYKMYGYGMPRVGNQAFADYIDANHPMTHVNNKEDYIPILPGMFLGFVHPSGEVHIEDSGSWDACPGQDNPSTLCIVGDVPEIWDGDESDHDGPYGPVTMGC

SEQ ID NO: 9 - protein, 280 aa, Bacillus macauensis
MKKTFVAVATAALLVTGFQGNASAEDDAQPPITAKWSAEDPHHEDTNTHLWIVRHAMEIMANNKDVVKPGEVEQLKQWQSDLEQGIYDADHANPYYDNATFASHFYDPDTGKSYIPLAAHAKTTSVKYFKRAGEAYQKGDHKQAFYNLGLALHYIGDLNQPMHAANFTNLSYPQGFHSKYENYVDSFKEDYAVKDGEGYWHWKGTNPEDWLHGTAVAAKKDYPDIVNDTTKAWFVKAAVSNSYAAKWRAAVVPATGKRLTEAQRILAGYMQLWFDTYVNK

SEQ ID NO: 10 - protein, 245 aa, Bacillus macauensis
WSAEDPHHEDTNTHLWIVRHAMEIMANNKDVVKPGEVEQLKQWQSDLEQGIYDADHANPYYDNATFASHFYDPDTGKSYIPLAAHAKTTSVKYFKRAGEAYQKGDHKQAFYNLGLALHYIGDLNQPMHAANFTNLSYPQGFHSKYENYVDSFKEDYAVKDGEGYWHWKGTNPEDWLHGTAVAAKKDYPDIVNDTTKAWFVKAAVSNSYAAKWRAAVVPATGKRLTEAQRILAGYMQLWFDTYVNK

SEQ ID NO: 11 - protein, 283 aa, Bacillus thuringiensis
MKKKVLALAAAITLVAPLQSVAFAHENDGGSKIKIVHRWSAEDKHKEGVNSHLWIVNRAIDIMSRNTTLVKQDRVAQLNEWRTELENGIYAADYENPYYDNSTFASHFYDPDNGKTYIPFAKQAKETGAKYFKLAGESYKNKDMKQAFFYLGLSLHYLGDVNQPMHAANFTNLSYPQGFHSKYENFVDTIKDNYKVTDGNGYWNWKGTNPEDWIHGAAVVAKQDYSGIVNDNTKDWFVKAAVSQEYADKWRAEVTPMTGKRLMDAQRVTAGYIQLWFDTYGDR

SEQ ID NO: 12 - protein, 245 aa, Bacillus thuringiensis
WSAEDKHKEGVNSHLWIVNRAIDIMSRNTTLVKQDRVAQLNEWRTELENGIYAADYENPYYDNSTFASHFYDPDNGKTYIPFAKQAKETGAKYFKLAGESYKNKDMKQAFFYLGLSLHYLGDVNQPMHAANFTNLSYPQGFHSKYENFVDTIKDNYKVTDGNGYWNWKGTNPEDWIHGAAVVAKQDYSGIVNDNTKDWFVKAAVSQEYADKWRAEVTPMTGKRLMDAQRVTAGYIQLWFDTYGDR

SEQ ID NO: 13 - protein, 320 aa, Pseudomonas sp
MTHLIGFAPRLLAFSALLLSQTAFSQESPAFIDPASWNTPFNGIAQVACHNCYEKQYANTFSSVLDSVRTLELDFWDQRDAVSGGSPHHWFVRHNPGTLFQSGNDNNCTGDGTGKNDLEACLNDVKNWSDKHPGHFPITLILDKKQGWSKESSGRTPKDFDELVARVFQGKLFTPQDLATHIGSGAGALQGNLKGKSWPTANDLQGKVLLVLNHSENQKLSQYAEARTSKAKVFISPVTNGQNDISGKVSGMSSQSSGYVAMNNMGKGDKSWAKQAFAYSHIGRVWGDDEVSFAQHINQKINLSAYYRFAAQSAGGYRIR

SEQ ID NO: 14 - protein, 297 aa, Pseudomonas sp
QESPAFIDPASWNTPFNGIAQVACHNCYEKQYANTFSSVLDSVRTLELDFWDQRDAVSGGSPHHWFVRHNPGTLFQSGNDNNCTGDGTGKNDLEACLNDVKNWSDKHPGHFPITLILDKKQGWSKESSGRTPKDFDELVARVFQGKLFTPQDLATHIGSGAGALQGNLKGKSWPTANDLQGKVLLVLNHSENQKLSQYAEARTSKAKVFISPVTNGQNDISGKVSGMSSQSSGYVAMNNMGKGDKSWAKQAFAYSHIGRVWGDDEVSFAQHINQKINLSAYYRFAAQSAGGYRIRPF

SEQ ID NO: 15 - protein, 610 aa, Penicillium emersonii
MRVLALIAALATVATASAPYDKRDLAQEIWDDIKNAVDCAGCQVVLTALKGVADLGTTALVDVLTEVCNISGKEDSDVCSGIISREGPVLDYVLQHLDIGSHTSQVICASAFGLCQYPEVRPYNLTFPKPKPNTTRPEPSGESPIQVVHFSDTHVDLSYETGSNYNCTKPICCRPYTAEDAPGNTTTPCGPYGNTKCDAPLSLEESMFAAIKALNPQPAFSIYTGDVVAHDIWLVDQNEVIEDLNATYDRMAGLGLVYAAIGNHDTAPVNDLPTSNIPSEYSANWTYEALSYDFTMLTQSASAQTAANYGSYSAIYPGSYGTDLRVISYNSIFYYVDNFWAYQDPMEFDPDGQLAWLINELQEAETAGQRVWIIAHVPTGTSDHFHDYSHYFDQIVQRYEATIAALFYGHTHIDQFQISYSNYSNRAFDTATAIGYIMPSLTPTSGPPTFRVYDVDPKTFAVLDFTNYIANISDPAFQSGPSWQKYYSAKETYGSLLSPPVTDPTAELTPAFWHNVTVAFEQDNATFQEYWARQTRGYDVSSCTGSCITQAICGLRAGDAQYNCVTPTPGFNFAKRDTSNPKQALSHVEKCEGSGLLGLLRRMVADSKSS

SEQ ID NO: 16 - protein, 594 aa, Penicillium emersonii
SAPYDKRDLAQEIWDDIKNAVDCAGCQVVLTALKGVADLGTTALVDVLTEVCNISGKEDSDVCSGIISREGPVLDYVLQHLDIGSHTSQVICASAFGLCQYPEVRPYNLTFPKPKPNTTRPEPSGESPIQVVHFSDTHVDLSYETGSNYNCTKPICCRPYTAEDAPGNTTTPCGPYGNTKCDAPLSLEESMFAAIKALNPQPAFSIYTGDVVAHDIWLVDQNEVIEDLNATYDRMAGLGLVYAAIGNHDTAPVNDLPTSNIPSEYSANWTYEALSYDFTMLTQSASAQTAANYGSYSAIYPGSYGTDLRVISYNSIFYYVDNFWAYQDPMEFDPDGQLAWLINELQEAETAGQRVWIIAHVPTGTSDHFHDYSHYFDQIVQRYEATIAALFYGHTHIDQFQISYSNYSNRAFDTATAIGYIMPSLTPTSGPPTFRVYDVDPKTFAVLDFTNYIANISDPAFQSGPSWQKYYSAKETYGSLLSPPVTDPTAELTPAFWHNVTVAFEQDNATFQEYWARQTRGYDVSSCTGSCITQAICGLRAGDAQYNCVTPTPGFNFAKRDTSNPKQALSHVEKCEGSGLLGLLRRMVADSKSS

SEQ ID NO: 17 - protein, 643 aa, Kionochaeta sp.
MRASSILSLALGLSVAQAAVNPADVLSVVEKRVDPASGLEVRSIWDTIWNDIKSAADCTACEAVLTLLKGVAAFGDNFFVEVLTEICDLSGAEDDDVCSGVLSLEGPIIANDIRKMSIGSKTSELFCITFLGLCSYPAVDAFTVPFPTAKSAATRPVSSGKDPIYVVHYSDIHIDPFYVAGSASNCTKPICCRDYTSASSPGNNNSPAGPYGDHNCDVPISLEDSMYAAIKKLVPDAAFGIFTGDIVDHAVWNTSESQNIIDMNDAYTRMKNSGMLPTIFATAGNHEASPVNSFPPPAIGNESQWVYDTLASDWSQWIGTSGASSVESIGAYSVQYGSTKLRVISLNTNMYYIENFYLYEPTMEQDPAGQFAWLVSELSAAEAAGERVWIIGHMPLGLSDAFHDPSNYFDQIVNRYEATIAAMFFGHTHEDHFQISYSDYNARTAANARAVSYIMPSLTPTSGHPTFRVYTVDPETFGVLDATTYYADMSQPTYQTAGPAWSVYYSAKAAYGGLVDPPVAADDAAAELTPAFWHNVTAALAADPASFDAYYARKTRGWDVAACAGACAAAEVCALRAARAQDNCVVPTPGVHFSKRADEGTLAHHRDECGVSVARNSLSSLVVQREALEHLEGRLSEKRRMAV

SEQ ID NO: 18 - protein, 625 aa, Kionochaeta sp.
AVNPADVLSVVEKRVDPASGLEVRSIWDTIWNDIKSAADCTACEAVLTLLKGVAAFGDNFFVEVLTEICDLSGAEDDDVCSGVLSLEGPIIANDIRKMSIGSKTSELFCITFLGLCSYPAVDAFTVPFPTAKSAATRPVSSGKDPIYVVHYSDIHIDPFYVAGSASNCTKPICCRDYTSASSPGNNNSPAGPYGDHNCDVPISLEDSMYAAIKKLVPDAAFGIFTGDIVDHAVWNTSESQNIIDMNDAYTRMKNSGMLPTIFATAGNHEASPVNSFPPPAIGNESQWVYDTLASDWSQWIGTSGASSVESIGAYSVQYGSTKLRVISLNTNMYYIENFYLYEPTMEQDPAGQFAWLVSELSAAEAAGERVWIIGHMPLGLSDAFHDPSNYFDQIVNRYEATIAAMFFGHTHEDHFQISYSDYNARTAANARAVSYIMPSLTPTSGHPTFRVYTVDPETFGVLDATTYYADMSQPTYQTAGPAWSVYYSAKAAYGGLVDPPVAADDAAAELTPAFWHNVTAALAADPASFDAYYARKTRGWDVAACAGACAAAEVCALRAARAQDNCVVPTPGVHFSKRADEGTLAHHRDECGVSVARNSLSSLVVQREALEHLEGRLSEKRRMAV

SEQ ID NO: 19 - protein, 632 aa, Nectria mariannaeae
MQLLSILAVGLGLAQNAFCQEVTHDLAGIKRSLESRDWVEDLWDKFESDATCAGCESLVLVLKGLAAISDQAFIDVLQEICKISGAEDDDVCDGSIQLEGPVIASGLRSMAIGSRTSKEFCTTFLGLCAYPAVQQWSVPFSSSKSSKTRPSSSGKDPIKVVHYSDIHIDPLYVGGSNSNCTKPICCRSYTKADQPGNNKYPAGPNGDHNCDSPVSLEKSMYNAIKEIVPDAAFTIFTGDIVDHAVWNTSQSYNTEQITNAYGLMSDNLGTIYGTAGNHEAHPANAFQPNSVGNVSQWVYDLLSGLWSQWISTEAKADSEKLGAYSTKYPGGNLRIISLNTNMYYRENYWLYRKTMIQDPSNQISWLVNELEAAETAGERVYIIGHMPLGDSNSFHDQSNYLDQVINRYSATISAMFFGHTHDDQFQISYSNWSNRNFSNALVTSYIGPSLTPTAGMPAFRVYDVDPVTFGILDSTTYIADMTDSAFQTTGPVWKKYYSAKEVYGSLLSPAVTDSSAELTAAFWHNVTTLFEADNTAFEAFLSRKSRGWKSESCTGTCKANEICQLRAARSENNCYTPSLGISFNKRSLNPVEERDECGISVTRATVSAMGVRKDVLRLLKKRFIEKAGEVRG

SEQ ID NO: 20 - protein, 596 aa, Nectria mariannaeae
DWVEDLWDKFESDATCAGCESLVLVLKGLAAISDQAFIDVLQEICKISGAEDDDVCDGSIQLEGPVIASGLRSMAIGSRTSKEFCTTFLGLCAYPAVQQWSVPFSSSKSSKTRPSSSGKDPIKVVHYSDIHIDPLYVGGSNSNCTKPICCRSYTKADQPGNNKYPAGPNGDHNCDSPVSLEKSMYNAIKEIVPDAAFTIFTGDIVDHAVWNTSQSYNTEQITNAYGLMSDNLGTIYGTAGNHEAHPANAFQPNSVGNVSQWVYDLLSGLWSQWISTEAKADSEKLGAYSTKYPGGNLRIISLNTNMYYRENYWLYRKTMIQDPSNQISWLVNELEAAETAGERVYIIGHMPLGDSNSFHDQSNYLDQVINRYSATISAMFFGHTHDDQFQISYSNWSNRNFSNALVTSYIGPSLTPTAGMPAFRVYDVDPVTFGILDSTTYIADMTDSAFQTTGPVWKKYYSAKEVYGSLLSPAVTDSSAELTAAFWHNVTTLFEADNTAFEAFLSRKSRGWKSESCTGTCKANEICQLRAARSENNCYTPSLGISFNKRSLNPVEERDECGISVTRATVSAMGVRKDVLRLLKKRFIEKAGEVRG

SEQ ID NO: 21 - DNA, 2889 bp, Rasamsonia eburnea
  source 1..2889: mol_type = genomic DNA
cggattagctgggtagacatagcctatcgcgtgtttgccctggggccacgaggacggctgtccctgaccaagattcaatcatcccgcgtcaggaggacacccgcttcgtgatgtttcattccagtcaatcattactgcactcttgcacagccaaaagccacgaccctgacgaatgacgaacgaacgcgctatctttttcgttcgttttcttttccacatagtagagcgttgatttagcgtccgactaagaagccaagcttttttgattgcttcatctacgatcctactctcaatgacacggcgcatcccccgcccgatcaacctcgggcacgtgggagtaaggggctccctgatcaattgatgatgcgttcaggctgtaataggggcaatatatagacatgggagggctaggctggctttgcatccgttctggaccctgggtatcccggcgagtccatctctgacaagttctgttcacttcaggccagtcaacaagacaaatgagagctttcctcatcacggcgctggcttcactagccaccgccgctggtgccacctatgacaagcgcggcctggcccaggatatctggaacgatatcaagaatgcggtggattgcgctggctgccagggcatcctgactgcattgaagggcctctcctatctaggcacgactgcctttgtcgacgtactgaccgaagtttgcgacatcagtggcgtacgtaggtgaatacccgtgcgcgtggatatactgactggattggctatcaggtggaagattcagatgtctgctcaggcatcatctccagcgagggtccggcgctggtctacatcctaaaacacctcgatatcggatcgcacacctcgcaggtcatctgtgctagtgtatttggtctctgccagtacccagcagtccgagcctacaacctcacgttcccttcgcccaagcccgacaagacttgtccagagcccagtggagaatcacccgtgcaaattgttcacttcagtgacacccacgcggatctctcgtacgagactggatccaattacaactgtaccaagcccatctgctgtcgctcgtacaccgcggaagacgctccgggaaacacgacgactccatgcggcccatatggcaaccccaagtgcgatgcccccatgagcctcgaggagagcatgtttgccgctatcaaagcgctgagcccgcagccagccttttccatctatacgggagacgtcgtcgcacatgacatctggcttgtggaccagaacgaagttgtcgaggatttgaatgctacctatgaccgcatggccggactgggactggtctacgccgcaattgggaaccacgacacggcgccggtcaacaatttgcctaccagcaacatccctagccagtacagcgcgaattggacgtacgaagcgcttgagtatcacttctcgctgctgaccaagtcggcgtccgcccagacagcagagaactacgggtcctattcctctgtctaccggggcagatacggaacggatctccgcgtaatttcctacaacagcatcttctattacatcgccgacttctgggcctaccaggaccccatgttgtacgacccagatgggcagctcgcctggctgatcaacgagctccaggaggccgaaacggcgggacaacgggtctggttgatagcgcatgttccgtcaggcacggccgaccatttccacgattactcgcactactttgaccagatcgtgcagcgctacgaggccaccattgctgcattgttctatggccacacgcacatcgatcagttccagatttcctactcggactactccaatcgagcgtttgacactgccacagccatcggctacatcatgccatcgatgacgccgacctcgggacctcctacctttcgagtttatgatgtcgatcccaagaccttcgcagtcttggacttcactaactatattgccaacatcagtgatccggcctaccagtccggcccgacgtggcagaagtactactcggcaaaggaggcgtacggatcattgctctctccgcccgtgacggacgcgacggcggagctgacgccagccttctggcacaacgtcacggtggcttttgagaacgatgacacggctttccaggagtactgggcgcggcagactcgcggatatgccgtgtcgagctgcacgggggactgcatcacgcaggcgatctgtggcttgcgggcgggagagtcgcagcacaactgcgtgacgccgacaccaggcttcaacttcgccaaacgagatgtttcgactgatggtcaggctttgccgcatattgagaaatgtgagggatcaggattgatggctttgctggccaagatggtggccagcaaccgacaatcttcctagttggaattgacggatacgagagtatatataagtgcagttaaagaattcagtcaacaatgtatagtacctagatttacaaagagtccatagtgtatgatgtacttattaacactatggcagtatagcggtgttggtgttttcaatagaaacgatgtggaaaactagaacattactatctcagaagatacagaaataatagggagtttcttctgggagttagaatatttggacccagaaagctccacttaatcagtgctgtatgtgcttggactcgaatagatcaataactaaaggtctatttaatatggctaagaatcgtgaaatccatatttatataataatattcacacatatagttatctttttaagtaatatatatgtaagaatataagagcagaggctaatcgatcggaggtactgtaattctcgttgatatttagactatgtaacaagctctgccctatttcctttcttctttttttttttttaacaatccctac

SEQ ID NO: 22 - protein, 611 aa, Rasamsonia eburnea
MRAFLITALASLATAAGATYDKRGLAQDIWNDIKNAVDCAGCQGILTALKGLSYLGTTAFVDVLTEVCDISGVEDSDVCSGIISSEGPALVYILKHLDIGSHTSQVICASVFGLCQYPAVRAYNLTFPSPKPDKTCPEPSGESPVQIVHFSDTHADLSYETGSNYNCTKPICCRSYTAEDAPGNTTTPCGPYGNPKCDAPMSLEESMFAAIKALSPQPAFSIYTGDVVAHDIWLVDQNEVVEDLNATYDRMAGLGLVYAAIGNHDTAPVNNLPTSNIPSQYSANWTYEALEYHFSLLTKSASAQTAENYGSYSSVYRGRYGTDLRVISYNSIFYYIADFWAYQDPMLYDPDGQLAWLINELQEAETAGQRVWLIAHVPSGTADHFHDYSHYFDQIVQRYEATIAALFYGHTHIDQFQISYSDYSNRAFDTATAIGYIMPSMTPTSGPPTFRVYDVDPKTFAVLDFTNYIANISDPAYQSGPTWQKYYSAKEAYGSLLSPPVTDATAELTPAFWHNVTVAFENDDTAFQEYWARQTRGYAVSSCTGDCITQAICGLRAGESQHNCVTPTPGFNFAKRDVSTDGQALPHIEKCEGSGLMALLAKMVASNRQSS

SEQ ID NO: 23 - protein, 552 aa, Rasamsonia eburnea
GLAQDIWNDIKNAVDCAGCQGILTALKGLSYLGTTAFVDVLTEVCDISGVEDSDVCSGIISSEGPALVYILKHLDIGSHTSQVICASVFGLCQYPAVRAYNLTFPSPKPDKTCPEPSGESPVQIVHFSDTHADLSYETGSNYNCTKPICCRSYTAEDAPGNTTTPCGPYGNPKCDAPMSLEESMFAAIKALSPQPAFSIYTGDVVAHDIWLVDQNEVVEDLNATYDRMAGLGLVYAAIGNHDTAPVNNLPTSNIPSQYSANWTYEALEYHFSLLTKSASAQTAENYGSYSSVYRGRYGTDLRVISYNSIFYYIADFWAYQDPMLYDPDGQLAWLINELQEAETAGQRVWLIAHVPSGTADHFHDYSHYFDQIVQRYEATIAALFYGHTHIDQFQISYSDYSNRAFDTATAIGYIMPSMTPTSGPPTFRVYDVDPKTFAVLDFTNYIANISDPAYQSGPTWQKYYSAKEAYGSLLSPPVTDATAELTPAFWHNVTVAFENDDTAFQEYWARQTRGYAVSSCTGDCITQAICGLRAGESQHNCVTPTPGFNFAK

SEQ ID NO: 24 - DNA, 2975 bp, Trichurus spiralis
  source 1..2975: mol_type = genomic DNA
gggggggggggggggggggggggaaagcggccgaggtccagtgcaggcggccctataccacccgctaatattccttatgatgatgacgaccttgctttttatctccaagctgagagcccttctcagctctggtgcttataaggctggcgggagggccgcggcttcgcgaacctgtcgatcttatttctcttacacgccaaatgctgctttgtatataacgggtggtctgtatatatacatatataaatagattctccgtgtgtatatacatcatagcatttatcccctcccgcgggtggcgctcttcaggcaatgcatctcactcgcgtcgcggccctcgcctttgggcttggcagggcgtttgccacggctcccgcgtctcagctcctctcgcgcgacgaggccgaactcctcggcaggggtctcgcggaagatgtctgggacgagatcaaggacggtctctcatgcgccggctgcgaagtacgatgcccctcaccatggccaactacccaccccctggacttaagtccacttacacgagcaacattgactaacaaccgccagaccctcgttgtcctgctcaagggcctcgcgctcttaggcgataacgcctttgtagatgtcctccaatcagtctgcaagctcacaaatgtaaaccccccgtttttgatccctcacccaaagagcttggatccgcatcaagactcacaaaaaactgacgttccctccttgctagagcgttgacgaggatgtctgccaaggcgccatagagctcgagggtcccatcatcgcagacgccatccggaacatgaaggtccccagccagacctccaagctcttctgcatcaacttcctcggcgtctgcccctacccgaaggtgacgccctaccaggtccccttccccacggagaagaaggccatcgagggcggctcgtcgcggcccgcggcgagcggcaaggacccgatccgcatcgtccactactccgacgtgcacgtcgaccacctttacacccccggctcgaccgcgaactgcacgaagcccatatgctgcaggccctacaccgaggacgacgagccgggcgtgagcacctcccccgccggccccaacggcgaccacaactgtgatacgcccgtgagcctcgaggagagcatgtacgccgccattaacgaggtcgccccggacgcggcctttgtcatttgcactggcgacatcgtcgaccacaccgtctggaacacctccgtcgagtacaacacattctctagtatgtccaccatgcccacctcccccgtccctctccaccccatctcatgtagcttctccgtatcgatcaagcgccaactactaacacgccggaacccccgccgccagtcaccgacgcctacagccgcatggctgcctccttcccgctcgtctacggcacggccggcaaccacgaggcccacccgacgaacgccttcccgcccacggccgaggccccgaccagctgggtctacaacctcctctctacgagctggtcccactggatcggcgacgccgccgcgtcctccacctccagccgcggctcctactctgtcaaataccccggcggtaacctgcgcgtcattagcatgaacacgaacaccttttacgtgcagaactactacctctaccgccgcgtcatgaccctcgacccggacggccagatcgcctggctcgcgtccgagctcgacgccgccgagaaggccggcgagagggcgtacatcatcggccacatgccccccggcgacaacgacgccttccacgaccagagcaactacttcgaccaggtcgtcaaccggtactccgacaccatcgctgccatgttcttcggccacacccacctcgacgagttccacgtcagctactcgtcctacgacgcccagaccgccgacaccgctgtcgccgtgcagtacgtcgccccgagcctcacgcccacgagcggccatccctcctttagggtttacgtcgtcgacccggagacctttgccatcctggacgccgagacctggatcgccgacatggccgaccccaacttccagactacccccacctggacgaagttctactccgccagggaagtctacgggcccgaccaccctgccggcgaagagctctcccccgccttctggcacaacgtcaccgaggccttcgcctcatcccccgacctcctggacgcctacctctcccggaagtcgcggggctgggaggacatcgcctgcgacgatgcttgtcgagagcaggagatttgcaagctacgtggcgggagggccgaggataactgctacgagccgaaacctggcatccatttcaagaagaggagcctggagtctaggggccatggggatgactgcggcgtctcggtgctgcgcggtgcggtgggttcgctcgctgtcgagaagaaggccgtggagcttgtgagtgcgagggtcgaggagaacaagagactcctaatctagaggcatcattagcgcttctcggtccgtaagagagagatggaggagggtggttggtagtgaagagggatacggatcttggatgaacagccaatttttattctggagctagaagttgtgctcgggatataggataaccaaaatcgctgtctctcttcttgaaaagctgaatcgctgtccgtctgtcttctcgggtaaccaaagtcgctggccgtcacttccgtggataaccaaagccgttgaccgtctccgtggataacattgtctatctccctccttacaccacccgagcttgatagcagtcattccatacgctagcaagcccgccctatcctcggtctgcagtacacaagctccaaaaaaaagaaatgatatcctagcgctcaaggcagcatcacattcctcccttcgcacctatgaatgctgcaatacaaactgattcgggcaacccactaacatggccggtaaaaaaaggctcgtcacaaaacctgccccgatgataa

SEQ ID NO: 25 - protein, 628 aa, Trichurus spiralis
MHLTRVAALAFGLGRAFATAPASQLLSRDEAELLGRGLAEDVWDEIKDGLSCAGCETLVVLLKGLALLGDNAFVDVLQSVCKLTNSVDEDVCQGAIELEGPIIADAIRNMKVPSQTSKLFCINFLGVCPYPKVTPYQVPFPTEKKAIEGGSSRPAASGKDPIRIVHYSDVHVDHLYTPGSTANCTKPICCRPYTEDDEPGVSTSPAGPNGDHNCDTPVSLEESMYAAINEVAPDAAFVICTGDIVDHTVWNTSVEYNTFSITDAYSRMAASFPLVYGTAGNHEAHPTNAFPPTAEAPTSWVYNLLSTSWSHWIGDAAASSTSSRGSYSVKYPGGNLRVISMNTNTFYVQNYYLYRRVMTLDPDGQIAWLASELDAAEKAGERAYIIGHMPPGDNDAFHDQSNYFDQVVNRYSDTIAAMFFGHTHLDEFHVSYSSYDAQTADTAVAVQYVAPSLTPTSGHPSFRVYVVDPETFAILDAETWIADMADPNFQTTPTWTKFYSAREVYGPDHPAGEELSPAFWHNVTEAFASSPDLLDAYLSRKSRGWEDIACDDACREQEICKLRGGRAEDNCYEPKPGIHFKKRSLESRGHGDDCGVSVLRGAVGSLAVEKKAVELVSARVEENKRLLI

SEQ ID NO: 26 - protein, 427 aa, Trichurus spiralis
ASGKDPIRIVHYSDVHVDHLYTPGSTANCTKPICCRPYTEDDEPGVSTSPAGPNGDHNCDTPVSLEESMYAAINEVAPDAAFVICTGDIVDHTVWNTSVEYNTFSITDAYSRMAASFPLVYGTAGNHEAHPTNAFPPTAEAPTSWVYNLLSTSWSHWIGDAAASSTSSRGSYSVKYPGGNLRVISMNTNTFYVQNYYLYRRVMTLDPDGQIAWLASELDAAEKAGERAYIIGHMPPGDNDAFHDQSNYFDQVVNRYSDTIAAMFFGHTHLDEFHVSYSSYDAQTADTAVAVQYVAPSLTPTSGHPSFRVYVVDPETFAILDAETWIADMADPNFQTTPTWTKFYSAREVYGPDHPAGEELSPAFWHNVTEAFASSPDLLDAYLSRKSRGWEDIACDDACREQEICKLRGGRAEDNCYEPKPGIHFKK

SEQ ID NO: 27 - DNA, 3204 bp, Trichoderma harzianum
  source 1..3204: mol_type = genomic DNA
tcctgatctgccgagccgcacaccatttcctctatttaactggtctctgccgatgcatgtgtgggagcctgcctctgattggatctccatctcggggcctcgctattagacggccctcagggaggccgttgaggggttcttactggacgatgcctcggtcctgcattcgttcagcgtggcattggagatggagcgagacagagtccggaaggtcatccgccctggggtgcgttcttgacttggcacagggccggttccttgtgagctgagtttcccttgtgaagggcccaaagttcgtatagttcgagcggagaggatttgcaacggcctggagtgcagttcgtagatgggtgggcactaacatcatgtaggtaggtggacgtggctatcgtctatttatatatagctatgatggaagacggtattcggttcctggttcctgatccttgcggacaacccaggcatctgaaggcagctcaagccagcatttgttaccagctatgcgtcccagctcgacgctctccctgctggctctgggctctgttgcccaggctgcggtttctcttgagaatagcagtctccctccccgagatatcgagaacatcgagagggctattgaggctagaagcttggctgatgatatatggaacgatatcaagaatgctgcgacatgcactgcttgccaggtcagaaaacaccattcacagtcatccgcatatgtgactctatgctgacacggctccagggcattctcgtccttctcaaaggcatcgctgtatttggagacggagcattcgttagcgtagctacggaactctgcaagctcgcaaaggtacctagatgactacgatggtctggttttcatctagccacggctaacgaaagtcttcaggttgaagacgatgatgtgtgcgaaggcaccgtaggactagagggtcccatcattgcggatgctatccgaaacatggatcttgggtctgatacatcaaaactcttctgtggatcgttccttggcctttgccctgagccctccgttcctcagtggaagattccttttccctcccccaagccacagactggtcgccctgcgccgagtgggaagacgcctctcaaggtggttcaatactcggatattcatattgaccctctttatgtgtctggctcaagctcgaattgcaccaagcccatctgctgcaggtgagtcgatttatacaaacaaagagtgacgatgaagtcaaaaagtaaagacactgacgatactgttactataaagaccttataccgccgctgacgagcctggcaagtcaacatcgcctgctgggccaaatggggaccacaagtgtgacactcctgtcggcttagaaattagcatgtatcaggccatcaagaacattgttccagatgctgccctaacgctattcacgggcgatattgttgaccatgcgatctggaacacctcgcagccttacaaccagaagcaaagtcagtgctcgcctttcttgaatatattgcgtatgagtattacgtagttgactaacatactccacttgcagtttccgacgcatacacttacatgagccagtatctgggcatcgtttacggtacggctggtaaccatgaatccaatcccacaaatgcttttcctcctaggtccatcagcaactcatcacaatgggtatatgatgccctatctgaccaatggacgcgctgggtgggggcctcagcagaatccgagattgaaagtatcggcgcatactctaccaagtatcccaatggcaacctgagagtcatctctctcaacaccaacttttactaccgcatgaacttttggctatatcaggagggtattgagcaggaccccgatggccaaatccagtggctcgtaagtgaactcgacgctgcggaaaaggcaggagagagagtttacatcattggacacatgcccctcggagaaggcgacgctttccacgcagggtcaaactaccttgatcaagttgtgaaccgctactcgtcgaccattgccgccatgttcttcggccacacacacgtcgatcactttgaggtcagctactccgactacaacagccgagatgcttcacatgctgtcatggcctcttacatctgcccctcgctgacgcccacctcgggcatgccctcgttccgagtatatgatgtcgacccggagacgtttgcagtgctcgacactacgacctacattgccgacatgaccaacgcggatttccaaacgactggccccgtgtggacgaagctttactctgccaaggagacgtacggttccaagctgaatccccccgtcaccgacgcaagtgtcgagctgactcccgcgttttggcacaacgtaacggccttgttcgactcaaactcggacgtgttcaacgagtacatctcgctcaagagccgtggctggaacgtcgcctcgtgcactggggattgcaagacgcaagagctctgccagcttcgagccggtcgtagcgagaacaactgcgtggttccttcaattggccttcaccttaacaagagatcggatgagctgcatgagcacgatcactcgcatcacggatcccacgaccaccaagaatgcggtatgtcggcaggtatgaagacgctgggctctttggctgtgaggaaggatttgttggatgagttggagactcgggctaatgagttgaaagccaaggcttgaggcatgagaaggttgtattttattcttgatagagcgtaatgaagacgtctttaatcccaattacgcgaggtaattgatttccggtcgaggtattatgtcatgctggaagctatgaaagcgagatctgtccgaacatcagcgaattcggaattgtatcatgaagtgaagccggagtctactaatggaggccgccggcgcaggcaaagtatggagtactgatgttcgtgctgccttttcaagcaaacttccaaggtacctgtacaattcatatgactagagataacgatgaggatctttttcagcttcatttcccggaagtcacagggatcaacaatgtattgcagtagaaacaaagatcttagatgatggagccgatgcgcgtctgctgtggctttgttgatattacgttcatgctccaagtcatgcacatgtacctctacccaatgctaccacttgacacaccaactgtctcgcagctgtaaatgtacaggtactgtccg

SEQ ID NO: 28 - protein, 645 aa, Trichoderma harzianum
MRPSSTLSLLALGSIAQAAVSLENSSLPRRDIENFERAIEARSLADDIWNDIKNAATCTACQGILVLLKGIAVFGDGAFVSVATELCKLAKVEDDDVCEGTVRLEGPIIADAIRNMDLGSDTSKLFCGSFLGLCPEPSVPQWKIPFPSPKPQTGRPAPSGKTPLKVVQYSDIHIDPLYVSGSSSNCTKPICCRPYTAADEPGKSTSPAGPNGDHKCDTPVGLEVSMYQAIKNIVPDAALTLFTGDIVDHAIWNTSQPYNQKQISDAYTYMSQYLGIVYGTAGNHESNPTNAFPPRSISNSSQWVYDALSDQWTRWVGASAESEIESIGAYSTKYPNGNLRVISLNTNFYYRMNFWLYQEGIEQDPDGQIQWLVSELDAAEKAGERVYIIGHMPLGEGDAFHAGSNYLDQVVNRYSSTIAAMFFGHTHVDHFEVSYSDYNSRDASHAVMASYICPSLTPTSGMPSFRVYDVDPETFAVLDTTTYIADMTNADFQTTGPVWTKLYSAKETYGSKLNPPVTDASVELTAAFWHNVTALFESNSDVFNEYISLKSRGWNVASCTGDCKTQELCQLRAGRSENNCVVPSIGLHLNKRSDGLHEHEHSHHRSHDHQECGMSAGMKTLGSLAVRKDLLDELETRANELKAKA

SEQ ID NO: 29 - protein, 521 aa, Trichoderma harzianum
SLADDIWNDIKNAATCTACQGILVLLKGIAVFGDGAFVSVATELCKLAKVEDDDVCEGTVGLEGPIIADAIRNMDLGSDTSKLFCGSFLGLCPEPSVPQWKIPFPSPKPQTGRPAPSGKTPLKVVQYSDIHIDPLYVSGSSSNCTKPICCRPYTAADEPGKSTSPAGPNGDHKCDTPVGLEISMYQAIKNIVPDAALTLFTGDIVDHAIWNTSQPYNQKQISDAYTYMSQYLGIVYGTAGNHESNPTNAFPPRSISNSSQWVYDALSDQWTRWVGASAESEIESIGAYSTKYPNGNLRVISLNTNFYYRMNFWLYQEGIEQDPDGQIQWLVSELDAAEKAGERVYIIGHMPLGEGDAFHAGSNYLDQVVNRYSSTIAAMFFGHTHVDHFEVSYSDYNSRDASHAVMASYICPSLTPTSGMPSFRVYDVDPETFAVLDTTTYIADMTNADFQTTGPVWTKLYSAKETYGSKLNPPVTDASVELTPAFWHNVTALFDSNSDVFNEYISLKSRGWNVASCTGDC

(19) United States
(12) Patent Application Publication
(10) Pub. No.: US 2023/0332069 A1
(43) Pub. Date: Oct. 19, 2023

(54) Method for Degumming and Refining of Vegetable Oil

(71) Applicant: Novozymes A/S, Bagsvaerd (DK)

(72) Inventors: Christian Hans Holm, Hellerup (DK); Per Munk Nielsen, Hillerod (DK); Fanny Longin, Frederiksberg (DK); Sara Maria Landvik, Vedbaek (DK); Jesper Brask, Vaerlose (DK); Kim Borch, Birkeroed (DK); Robert Piotr Olinski, Vaerlose (DK); Allan Noergaard, Lyngby (DK); Hanna Maria Lilbaek, Copenhagen (DK); Marianne Linde Damstrup, Copenhagen (DK); Tianqi Sun, Beijing (CN); Ming Li, Beijing (CN)

(73) Assignee: Novozymes A/S, Bagsvaerd (DK)

(21) Appl. No.: 18/055,373

(22) Filed: Nov. 14, 2022

Related U.S. Application Data
(63) Continuation of application No. 16/491,093, filed on Sep. 4, 2019, now abandoned.

(30) Foreign Application Priority Data
Mar. 20, 2017 (WO) PCT/CN2017/077326

(51) Int. Cl. C11B 3/00; C12N 9/18

(52) U.S. Cl. CPC C11B 3/003; C12N 9/18

(57) ABSTRACT

The present invention relates to refining of vegetable oil. The invention provides processes in which phospholipids present in the vegetable oil are hydrolysed and the oil is subsequently subject to chemical refining.

### REFERENCE TO A SEQUENCE LISTING

This application contains a Sequence Listing in computer readable form, which is incorporated herein by reference.

### FIELD OF THE INVENTION

The present invention relates to methods for degumming and refining vegetable oil. The invention further relates to polypeptides having phospholipase A activity, to polypeptides having phospholipase C activity and to polynucleotides encoding the polypeptides. The invention also relates to nucleic acid constructs, vectors, and host cells comprising the polynucleotides as well as methods of producing and using the polypeptides.

### BACKGROUND OF THE INVENTION

Whether intended for human consumption or as feedstock in production of oleo chemicals or biodiesel, vegetable oil needs to be pretreated to remove impurities, such as phospholipids (“gums”) and free fatty acids. The pretreatment includes Degumming, Refining (also referred to a “Neutralization”, Bleaching and Deodorization).

The purpose of the degumming process is to remove hydratable and non-hydratable phospholipids or gums present in the oil. Traditionally, the degumming process has been based on use of water extraction (“water degumming”), which involves treating the oil with water and separation of the hydratable phospholipids or gums from the triglyceride oil. Depending on the source of oil, water degumming may be combined with “acid degumming” in which the oil is treated with acid the non-hydratable gums are separated from the triglyceride oil.

Enzymatic degumming is performed on oils which have been water degummed as well as on crude oils. In the enzymatic degumming process, the phospholipids are hydrolysed in a reaction catalyzed by enzymes having phospholipase activity and are thereby converted into water soluble and water extractable components.

There are two general types of refining: “Chemical refining” (also referred to as “Alkali refining”) and “Physical refining”. Chemical refining, which comprises treatment of the oil with an alkali solution or other refining solution, is performed to reduce the free fatty acid content and will also remove other impurities such as phospholipids, proteinaceous and mucilaginous substances and color compounds. This process results in a large reduction of free fatty acids through their conversion into high specific gravity soaps, which are removed by centrifugation with some loss of neutral oil. Most phosphatides and mucilaginous substances are soluble in the oil only in an anhydrous form and upon hydration with the caustic or other refining solution are readily separated. After alkali refining, the fat or oil is water-washed to remove residual soap.

Oils low in phospholipid content (palm and coconut) may be physically refined (i.e. steam stripped) to remove free fatty acids. In physical refining, free fatty acids in crude or water degummed oil are removed by evaporation rather than being neutralized and removed as soap in an alkaline refining process.

Although enzymatic degumming has recently become more widespread, it has never been accepted by the industry as a process integrated with the chemical refining. The concern has been that production of too much FFA would lead to undesired increase of soap formation in the refining, or that enzyme technology would not be compatible at the pH conditions in chemical refining: In chemical refining the first stage is an acid chelating step followed by high pH conditions from the alkaline addition. Hence, chemical refining is typically applied to crude oil or, as shown in FIG. 2 herein, to oil that has been subject to water degumming and/or acid degumming. The skilled person will also know that in conventional processes, chemical refining is associated with considerable yield loss if the hydratable and non-hydratable gums are not removed prior to application of the caustic or other refining agent(s).

Despite recent advances in oil degumming and refining, there is a need for providing novel simplified methods for degumming and refining of vegetable oil in which the oil loss is minimized. The present invention relates to such novel methods, to novel polypeptides having phospholipase A activity, to novel polypeptides having phospholipase C activity and to polynucleotides encoding the polypeptides.

### SUMMARY OF THE INVENTION

Contrary to what was previously believed, the inventors have observed that when refining a vegetable oil containing phospholipids, considerable advantages are provided when the phospholipids are subject to enzymatic hydrolysis and the oil is subsequently subject to chemical refining without separation of gum phase in between the hydrolysis and the refining step. In particular, the inventors observed a significant yield increase as compared to performing chemical refining on crude oil or on oil that had been subject to water degumming.

Accordingly, the present invention provides in a first aspect a method for refining a vegetable oil containing phospholipids, comprising subjecting the phospholipids to enzymatic hydrolysis by contacting the vegetable oil with one or more phospholipid degrading enzymes, and thereafter subjecting the vegetable oil to chemical refining.

In a second aspect the invention relates to the use of a phospholipid degrading enzyme to hydrolyze phospholipids in a vegetable oil, wherein the vegetable oil is contacted with the phospholipid degrading enzyme, and thereafter subjected to chemical refining.

In a third aspect the invention provides a refined vegetable oil or a soapstock, which is obtainable or is obtained by the method according to the invention.

In a fourth aspect, the invention relates to an isolated or purified polypeptide having phospholipase A activity, selected from the group consisting of:
a. A polypeptide having at least 75% sequence identity, such as at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of any one of SEQ ID NOs: 3 and 5,
b. A polypeptide having at least 75% sequence identity, such as at least 80%, at least 85%, at least 90%, at least 91%, at least 92%, at least 93%, at least 94%, at least 95%, at least 96%, at least 97%, at least 98%, at least 99% or 100% sequence identity to the polypeptide set forth in any one of SEQ ID NOs: 4 and 6;
c. A fragment of the polypeptide of (a) or (b), that has phospholipase A activity.

In a fifth aspect the invention provides an isolated or purified polypeptide having phospholipase C activity, selected from the group consisting of:
i) A polypeptide having at least 60% sequence identity to the mature polypeptide of any one of SEQ ID NOs: 19, 21, 23,
ii) A polypeptide having at least 60% sequence identity to the polypeptide set forth in any one of SEQ ID NOs: 20, 22, 24: and
iii) A fragment of the polypeptide of (a) or (b) that has phospholipase C activity.

In a sixth aspect, the invention provides a composition comprising the polypeptide according to the invention.

In a seventh aspect, the invention provides an isolated or purified polynucleotide encoding the polypeptide of the invention.

In an eight aspect the invention relates to a nucleic acid construct or expression vector comprising the polynucleotide of the invention, wherein the polynucleotide is preferably operably linked to one or more control sequences that direct the production of the polypeptide in an expression host.

In a ninth aspect, the invention relates to a recombinant host cell comprising the polynucleotide of the invention, operably linked to one or more control sequences that direct the production of the polypeptide.

In a tenth aspect, the invention provides a method of producing the polypeptide of the invention, comprising cultivating a cell, which in its wild-type form produces the polypeptide, under conditions conducive for production of the polypeptide.

In an eleventh aspect, the invention relates to a method of producing a polypeptide having phospholipase A activity or a polypeptide having phospholipase C activity, comprising cultivating the recombinant host cell of the invention under conditions conducive for production of the polypeptide.

### BRIEF DESCRIPTION OF THE DRAWINGS

The text Lipr287 refers to Bacillus macauensis PLC: Mature polypeptide of SEQ ID NO: 9 FIG. 1 illustrates where different phospholipases cleave a phospholipid as well as the four major functional groups on phospholipids.

FIG. 2 illustrates processes for treatment of vegetable oil, including degumming and refining.

FIG. 3 shows yield estimate after end centrifugation. Mature polypeptide of SEQ ID NO: 9 as pre-treatment for alkaline degumming; 70° C., 1 hour enzyme reaction, 3% total water, 1141 ppm NaOH total.

FIG. 4 shows delta diglyceride content. Mature polypeptide of SEQ ID NO: 9 as pre-treatment for alkaline degumming; 0° C., 1 hour enzyme reaction, 3% total water, 1141 ppm NaOH total.

FIG. 5 shows Intact phospholipids. Mature polypeptide of SEQ ID NO: 9 as pre-treatment for alkaline degumming; 70° C., 1 hour enzyme reaction, 3% total water, 1141 ppm NaOH total.

FIG. 6 shows hydrolyzed phospholipids (all 4). Mature polypeptide of SEQ ID NO: 9 as pre-treatment for alkaline degumming; 70° C., 1 hour enzyme reaction, 3% total water, 1141 ppm NaOH.

FIG. 7 shows hydrolyzed PC+PE. Mature polypeptide of SEQ ID NO: 9 as pre-treatment for alkaline degumming; 70° C., 1 hour enzyme reaction, 3% total water, 1141 ppm NaOH total.

FIG. 8 shows total phosphorous content after end centrifugation. Mature polypeptide of SEQ ID NO: 9 as pre-treatment for alkaline degumming; 70° C., 1 hour enzyme reaction, 3% total water, 1141 ppm NaOH total.

FIG. 9 shows total oil after end centrifugation; 650 ppm Ca, 2.5% total water, 3000 ppm NaOH for alkaline treatment at 70° C.

FIG. 10 shows Yield gain compared to blank. 650 ppm Ca, 2.5% total water, 3000 ppm NaOH for alkaline treatment at 70° C. in low NHP oil (15 ppm P).

FIG. 11 shows FFA content before and after alkaline treatment; 650 ppm Ca, 2.5% total water, 3000 ppm NaOH for alkaline treatment at 70° C.

FIG. 12 shows delta di-glyceride content; 650 ppm Ca, 2.5% total water, 3000 ppm NaOH for alkaline treatment at 70° C.

### DETAILED DESCRIPTION OF INVENTION

### Definitions

Alkali: In the present context “alkali” refers interchangeably to a base that is soluble in water and forms hydroxide ions, such as NaOH, KOH, sodium carbonate, Ca(OH)₂, and Mg(OH)₂ and to the solution of a base in water.

Bleaching: The term “bleaching” refers to the process for removing color producing substances and for further purifying the fat or oil. Normally, bleaching is accomplished after the oil has been refined.

Chemical refining: In the present application, the term “chemical refining” is used synonymously with “alkali refining” and “alkaline refining”; the term also covering “caustic refining” and “caustic neutralization”.

Crude oil: The term “crude oil” refers to a pressed or extracted unrefined and unprocessed oil from a vegetable source, including but not limited to acai oil, almond oil, babassu oil, blackcurrent seed oil, borage seed oil, canola oil, cashew oil, castor oil, coconut oil, coriander oil, corn oil, cottonseed oil, crambe oil, flax seed oil, grape seed oil, hazelnut oil, hempseed oil, jatropha oil, jojoba oil, linseed oil, macadamia nut oil, mango kernel oil, meadowfoam oil, mustard oil, neat's foot oil, olive oil, palm oil, palm kernel oil, palm olein, peanut oil, pecan oil, pine nut oil, pistachio oil, poppy seed oil, rapeseed oil, rice bran oil, safflower oil, sasanqua oil, sesame oil, shea butter, soybean oil, sunflower seed oil, tall oil, tsubaki oil walnut oil, varieties of “natural” oils having altered fatty acid compositions via Genetically Modified Organisms (GMO) or traditional “breading” such as high oleic, low linolenic, or low saturated oils (high oleic canola oil, low linolenic soybean oil or high stearic sunflower oils). The term also encompasses a mixture of several pressed or extracted unrefined and unprocessed oils from sources as defined above.

Deodorization: “Deodorization” is a vacuum steam distillation process for the purpose of removing trace constituents that give rise to undesirable flavors, colors and odors in fats and oils. Normally this process is accomplished after refining and bleaching.

Fractionation: Fractionation is the process of separating the triglycerides in fats and oils by difference in melt points, solubility or volatility. It is most commonly used to separate fats that are solid at room temperature but is also used to separate triglycerides found in liquid oils.

Gum: In the context of the present invention “gum”, “gums” or “gum fraction” refers to a fraction enriched in phosphatides, which is separated from the bulk of vegetable oil during a degumming process. “Gums” consist mainly of phosphatides but also contain entrained oil, contain nitrogen and sugar and meal particles

Heterologous: The term “heterologous” means, with respect to a host cell, that a polypeptide or nucleic acid is not naturally occurring in a host cell. The term “heterologous” means, with respect to a polypeptide or nucleic acid, that a control sequence, e.g., promoter, or domain of a polypeptide or nucleic acid is not naturally associated with the polypeptide or nucleic acid, i.e., the control sequence is from a gene other than the gene encoding the polypeptide of SEQ ID NO: 1.

Host cell: The term “host cell” means any microbial or plant cell into which a nucleic acid construct or expression vector comprising a polynucleotide of the present invention has been introduced. Methods for introduction include but are not limited to protoplast fusion, transfection, transformation, electroporation, conjugation, and transduction. In some embodiments, the host cell is an isolated recombinant host cell that is partially or completely separated from at least one other component with, including but not limited to, for example, proteins, nucleic acids, cells, etc.

Isolated: The term “isolated” means a polypeptide, nucleic acid, cell, or other specified material or component that is separated from at least one other material or component with which it is naturally associated as found in nature, including but not limited to, for example, other proteins, nucleic acids, cells, etc. An isolated polypeptide includes, but is not limited to, a culture brother containing the secreted polypeptide.

Lysophospholipase: A “lysophospholipase” (EC 3.1.1.5) is an enzyme that can hydrolyze 2-lysophospholids to release fatty acid.

Lysophospholipase activity (LLU) may be measured using egg yolk L-α-lysolecithin as the substrate with a NEFA C assay kit. 20 μl of sample is mixed with 100 μl of 20 mM sodium acetate buffer (pH 4.5) and 100 μl of 1% L-α-lysolecithin solution, and incubated at 55° C. for 20 min. After 20 min, the reaction mixture is transferred to the tube containing 30 μl of Solution A in NEFA kit preheated at 37° C. After 10 min incubation at 37° C., 600 μl of Solution B in NEFA kit is added to the reaction mixture and incubated at 37° C. for 10 min. Activity is measured at 555 nm on a spectrophotometer. One unit of lysophospholipase activity (1 LLU) is defined as the amount of enzyme that can increase the A550 of 0.01 per minute at 55° C.

Mature polypeptide: The term “mature polypeptide” means a polypeptide in its final form following translation and any post-translational modifications, such as N-terminal processing, C-terminal truncation, glycosylation, phosphorylation, and removal of signal peptides, propeptides and prepropeptides. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Phospholipase A activity: In the context of the present invention the term “phospholipase A activity” comprises enzymes having phospholipase A1 and/or phospholipase A2 activity (A1 or A2, EC 3.1.1.32 or EC 3.1.1.4), i.e., hydrolytic activity towards one or both carboxylic ester bonds in phospholipids such as lecithin. A phospholipases having both A1 and A2 activity is also referred to as a phospholipase B.

For purposes of the present invention, phospholipase A activity is preferably determined according to the following procedure.

Phospholipase a Activity (LEU)

In the LEU assay, the phospholipase A activity is determined from the ability to hydrolyze lecithin at pH 8.0, 40° C. The hydrolysis reaction can be followed by titration with NaOH for a reaction time of 2 minutes. The phospholipase from Fusarium oxysporum (LIPOPAN F) disclosed in WO 1998/26057 has an activity of 1540 LEU/mg enzyme protein and may be used as a standard.

Plate Assay
A) Buffers is a mixture of 100 mM HEPES and 100 mM Citrate with pH adjusted from pH 3.0 to pH 7.0.
B) 2% Agarose (Litex HSA 1000) is prepared by mixing and cooking in buffers (A)) for 5 minutes followed by cooling to approximately 60° C.
C) Substrate is L-alfa Phosohatidylcholine, 95% from Soy (Avanti 441601) dispersed in water (MilliQ) at 60° C. for 1 minute with Ultra Turrax.
D) Purified enzyme solutions of LECITASE ULTRA and the mature phospholipase of SEQ ID NO: 2 were diluted to 0.4 mg/ml.

Plates are casted by mixing of 5 ml substrate (C)) and 5 ml Agarose (B)) gently mixed into petri dishes with diameter of 7 cm and cooled to room temperature before holes with a diameter of approximately 3 mm are punched by vacuum. Ten microliters diluted enzyme (D)) is added into each well before plates are sealed by parafilm and placed in an incubator at 55° C. for 48 hours. Plates are taken out for photography at regular intervals.

Phospholipase activity: In the context of the present invention, the term “phospholipase activity” refers to the catalysis of the hydrolysis of a glycerophospholipid or glycerol-based phospholipid.

Conditions facilitating hydrolysis of phospholipids: Selecting the conditions which will facilitate hydrolysis of phospholipids by phospholipid degrading enzymes is within the skill of a person skilled in the art, and includes for example adjusting pH, and/or temperature at which phospholipid degrading enzyme are active.

Phospholipase C activity: The term “phospholipase C activity” or “PLC activity” relates to an enzymatic activity that removes the phosphate ester moiety from a phospholipid to produce a 1,2 diacylglycerol (see FIG. 1). Most PLC enzymes belong to the family of hydrolases and phosphodiesterases and are generally classified as EC 3.1.4.3, E.C. 3.1.4.11 or EC 4.6.1.13. Phospholipase C activity may be determined according to the procedure described in the following Phospholipase C assay:

Phospholipase C activity assay: Reaction mixtures comprising 10 microL of a 100 mM p-nitrophenyl phosphoryl choline (p-NPPC) solution in 100 mM Borax-HCl buffer, pH 7.5 and 90 microL of the enzyme solution are mixed in a microtiter plate well at ambient temperature. The microtiter plate is then placed in a microtiter plate reader and the released p-nitrophenol is quantified by measurement of absorbance at 410 nm. Measurements are recorded during 30 min at 1 minute intervals. Calibration curves in the range 0.01-1 microL/ml p-nitrophenol are prepared by diluting a 10 micromol/ml p-nitrophenol stock solution from Sigma in Borax-HCl buffer. One unit will liberate 1.0 micromol/minute of p-NPPC at ambient temperature.

Phospholipase C specificity: The term “phospholipase C specificity” relate to a polypeptide having phospholipase C activity where the activity is specified towards one or more phospholipids, with the four most important once being phosphatidylcholine (PC), phosphatidylethanolamine (PE), phosphatidic acid (PA) and phosphatidyl inositol (PI) (see FIG. 1). Phospholipase C specificity may be determined by 31P-NMR as described above in relation to the term “phospholipase activity”.

PC and PE-specific phospholipase C: The terms “PC and PE-specific phospholipase C” and “phospholipase C having specificity for phosphatidyl choline (PC) and phosphatidyl ethanolamine (PE)” and “polypeptide having activity towards phosphatidylcholine (PC) and phosphatidylethanolamine (PE)” are used interchangeably. They relate to a polypeptide having activity towards phosphatidylcholine (PC), phosphatidylethanolamine (PE). In addition to the PC and PE specificity it may also have some activity towards phosphatidic acid (PA) and phosphatidyl inositol (PI). Preferably a PC and PE specific phospholipase C removes at least 30% PC and at least 30% PE from an oil or fat with at least 100 ppm PC and 100 ppm PE when using the P-NMR assay of Example 1 at the optimal pH of the enzyme and an enzyme dosage of 10 mg/kg. More preferably it removes 40%, 50%, 60%, 70% or 80%, even more preferred it removes 90% and most preferred it removes between 90% and 100% of the PC in the oil or fat and 40%, 50%, 60%, 70% or 80%, even more preferred it removes 90% and most preferred it removes between 90% and 100% of the PE in the oil or fat.

PI-Specific Phospholipase C: The terms “PI-specific phospholipase C”, “Phosphatidylinositol phospholipase C” and “polypeptide having activity towards phosphatidylinositol (PI)” are used interchangeably. They relate to a polypeptide having activity towards phosphatidyl inositol (PI), meaning that its activity towards phosphatidylcholine (PC), phosphatidylethanolamine (PE), phosphatidic acid (PA) is low compared to the PI activity. PI-specific phospholipase C enzymes can either belong to the family of hydrolases and phosphodiesterases classified as EC 3.1.4.11 or to the family of lyases classified as EC 4.6.1.13. PI-specific phospholipase C activity may be determined according to the procedure described in Example 5. Preferably a PI-specific phospholipase C removes at least 30% PI from an oil or fat with at least 50 ppm PI when using the P-NMR assay of Example 1 at the optimal pH of the enzyme and an enzyme dosage of 10 mg/kg. More preferably it removes 40%, 50%, 60%, 70% or 80%, even more preferred it removes 90% and most preferred it removes between 90% and 100% of the PI in the oil or fat.

Preferably a PI-specific Phospholipase C removes at least 20% more PI when compared to the amount of PC, PE or PA it can remove, more preferred at least 30%, 40%, even more preferred at least 50% and most preferred at least 60% more PI when compared to the amount of PC, PE or PA it can remove.

PC-, PE-, PA- and PI-Specific Phospholipase C: The terms “PC-, PE-, PA,- and PI-specific phospholipase C”, and “polypeptide having activity towards phosphatidylcholine (PC), phosphatidylethanoamine (PE), phosphatidic acid (PA) and phosphatidylinositol (PI)” are used interchangeably. They relate to a polypeptide having activity towards phosphatidylcholine (PC), phosphatidylethanoamine (PE), phosphatidic acid (PA), and phosphatidyl inositol (PI). Preferably a PC-, PE-, PA,- and PI-specific phospholipase C removes at least 30% of each of the four phospholipid species from an oil or fat with at least 100 ppm PC, 75 ppm PE, 5 ppm PA and 50 ppm PI when using the P-NMR assay of Example 1 at the optimal pH of the enzyme and an enzyme dosage of 10 mg/kg. More preferably it removes 40%, 50%, 60%, 70% or 80%, even more preferred it removes 90% and most preferred it removes between 90% and 100% of the PC in the oil or fat and 40%, 50%, 60%, 70% or 80%, even more preferred it removes 90% and most preferred it removes between 90% and 100% of the PE in the oil or fat.

Purified: The term “purified” means a nucleic acid or polypeptide that is substantially free from other components with which it is associated in nature, as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

Recombinant: The term “recombinant,” when used in reference to a subject cell, nucleic acid, protein or vector, indicates that the subject has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a polypeptide is a recombinant vector. The term “recombinant” is synonymous with “genetically modified” and “transgenic”.

Reaction rate: For the purpose of the present invention “reaction rate” is synonymous with “rate of reaction” and is defied according to IUPAC, Compendium of Chemical Terminology, 2^{nd} ed. (the “Gold Book”) (1997): “Rate of reaction”.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the—nobrief option must be specified in the command line. The output of Needle labeled “longest identity” is calculated as follows:

(Identical Residues×100)/(Length of Alignment−Total Number of Gaps in Alignment)

Soapstock: In the present contexts, “soapstock” refers to a fraction containing soaps, which is separated from the bulk of vegetable oil during a chemical refining process. The soaps are formed by reaction of a refining chemical, such as alkaline, with free fatty acids in the present in the vegetable oil. The exact compostion of soapstocks depends on the vegetable oil source from which they are obtained; cottonseed soapstock, for instance, was found to be mainly composed of moisture and solvent, fatty acids, organic phosphates, monoglycerides, diglycerides, triglycerides, sterols, polyalcohols, carbohydrates and other miscellaneous components. The majority of these classes of organic compounds are found in soapstocks from other vegetable oils.

Stoichiometric amount: The term “Stoichiometric amount” means, in effect, the measure of amount required for stoichiometry; i.e. the optimum amount where, assuming that the reaction proceeds to completion, all of the reagent is consumed, there is no deficiency of the reagent, and there is no excess of the reagent.

In the context of the invention “stoichiometric amount” refers in particular to the number of moles of a reagent (e.g. alkali, such as NaOH) added to a reaction mixture, which is equal to the number of moles of the compounds (e.g. free fatty acids and/or acid added as calcium chelating agent, such as citric acid) with which the reagent reacts in said reaction mixture.

Water degumming: The term “water degumming” refers to a process which involves treating crude oil with an amount of water to hydrate phospholipids present in the oil and make them separable by centrifugation.

In a first aspect, the present invention provides a method for refining a vegetable oil containing phospholipids, comprising subjecting the phospholipids to enzymatic hydrolysis by contacting the vegetable oil with one or more phospholipid degrading enzymes under conditions facilitating hydrolysis of phospholipids, and thereafter subjecting the vegetable oil to chemical refining.

In preferred embodiments of the invention, there is no or little separation of the reaction products of the enzymatic hydrolysis from the oil, prior to said chemical refining. Hence, the enzymatic hydrolysis of the phospholipids may be performed in a first reaction vessel and the chemical refining may be performed in a second reaction vessel, the two reaction vessels being fluidly connected and/or being connected so as to allow liquid passage from the first to the second reaction vessel.

In further preferred embodiments, the enzymatic hydrolysis of the phospholipids and the chemical refining are performed in the same vessel. That is, the enzymatic hydrolysis of the phospholipids is performed in a reaction vessel, and the chemical refining is performed after the enzymatic hydrolysis, in the same reaction vessel. Such embodiments avoid the need to transfer the contents of a first reaction vessel to a second reaction vessel, as both reactions take place in one and the same reaction vessel.

In further embodiments, the chemical refining is performed simultaneously with, or subsequent to the enzymatic hydrolysis, preferably subsequent to enzymatic hydrolysis.

The method of the invention may be performed as a batch process, or as a continuous process. Thus the process can fit into existing process setup whether it is a batch operation or the typical continuous process used in the industry. One particular embodiment relates to the method according to the invention wherein the chemical refining is performed immediately after the enzymatic hydrolysis; preferably in a continuous process operation.

It is further to be understood that the enzymatic hydrolysis of the phospholipids may be performed in a first reaction vessel and the chemical refining may be performed in a second reaction vessel, wherein fluid connection between the reaction vessels or liquid passage from the first to the second reaction vessel is not via a separation device, such as a centrifuge.

In some embodiments, the method according to the invention is one, wherein
the enzymatic hydrolysis is performed in a reaction mixture comprising a heavy phase, or aqueous phase, and a light phase, or oil phase/hydrophobic phase, and
there is no reduction or no substantial reduction of the heavy phase volume or separation of gums/heavy phase from oil before said chemical refining.

In conventional degumming the two phases are mixed, e.g. by use of a high shear mixer, and an emulsion is created. In the emulsion, the enzyme reacts with the phospholipids to produce water soluble reaction products. The emulsion is the broken, e.g. by centrifugation, separating the water soluble reaction products from the oil. The method according to the invention preferably does not include any step to separate the heavy phase/aqueous phase or part thereof containing water soluble reaction products, from the light phase, oil phase or hydrophobic phase.

Preferably, the enzymatic hydrolysis is performed by contacting said vegetable oil with one or more enzymes having phospholipase activity.

The method according to the invention may comprise
i) Providing a reaction mixture comprising said vegetable oil and the one or more enzymes having phospholipid degrading activity, such as a reaction mixture comprising a heavy phase, or aqueous phase, and a light phase, or oil phase/hydrophobic phase;
ii) Subjecting the reaction mixture to conditions allowing enzymatic hydrolysis of phospholipids in the oil, to provide a reacted mixture of said vegetable oil; and
iii) Subjecting the reacted mixture of said vegetable oil to chemical refining.

The method according to the invention may further comprise subjecting the vegetable oil to water degumming before contacting it with the one or more phospholipid degrading enzymes.

In further embodiments, the vegetable oil is selected from the group consisting of acai oil, almond oil, babassu oil, blackcurrent seed oil, borage seed oil, canola oil, cashew oil, castor oil, coconut oil, coriander oil, corn oil, cottonseed oil, crambe oil, flax seed oil, grape seed oil, hazelnut oil, hempseed oil, jatropha oil, jojoba oil, linseed oil, macadamia nut oil, mango kernel oil, meadowfoam oil, mustard oil, neat's foot oil, olive oil, palm oil, palm kernel oil, palm olein, peanut oil, pecan oil, pine nut oil, pistachio oil, poppy seed oil, rapeseed oil, rice bran oil, safflower oil, sasanqua oil, sesame oil, shea butter, soybean oil, sunflower seed oil, tall oil, tsubaki oil and walnut oil.

In preferred embodiments of the invention the vegetable oil is selected from the group consisting of rapeseed oil, soybean oil, sunflower seed oil, palm oil, coconut oil, rice bran oil and peanut oil/ground nut oil. These vegetable oils are, from a commercial point of view, considered important as they are abundant and large volumes of the oil are processed to meet consumers preferences for very light colored cooking oil or are used as feedstock for biofuel production.

In some of the embodiments of the invention, the vegetable oil, which is contacted with said one or more phospholipid degrading enzymes is a crude vegetable oil.

The method according to the invention may comprise contacting the vegetable oil with one or more chelation agents capable of complexing Ca and/or Mg ions prior to contacting it with the one or more phospholipid degrading enzymes. Suitable chelation agents may be selected from the group consisting of citric acid, phosphoric acid, lactic acid and ethylenediaminetetraacetic acid (EDTA).

The reaction mixture may have a pH, which is in the range of 1.5-7. As the skilled person will understand, the requirements for adjustment of pH depends on the requirement of the enzyme(s) used and on the amounts of any chelating agent that has been added. In particular, the pH may be within the range of 3-7, such as 3.5-6.6, within the range of 3-5, such as 3.5-4.5, or within the range of 5-7, such as 4.5-6.5.

In one embodiment, the pH is adjusted by addition of base, for example by addition of NaOH, KOH, sodium carbonate or combinations thereof. In particular embodiments, the amount of equivalents of base used to neutralize the acid of the pretreatment is in the range of from 1.2 to 7 equivalents, such as from 1.5 to 6, 1.5 to 5 equivalents; or for example 2 to 7, 3 to 7 or such as 3 to 7 or 3 to 5 equivalents to the acid; in further particular, the one or more phospholipid degrading enzymes comprise or consist of SEQ ID NO. 11 and SEQ ID NO. 13.

In the method according to the invention, the reaction mixture has a water content in the range of 0.5-10% (w/w), such as in the range of 1-10% (w/w), in the range of 1-5% (w/w)—such as in the range of 0.5-5% (w/w), such as a water content of 5% (w/w) or less, such as a water content of 4% or less or such as a water content of 3% or less.

The vegetable oil is contacted with the one or more phospholipid degrading enzymes at a temperature, which is in the range of 45-90° C., such as in the range of 50-90° C., 60-90° C., 60-80° C., 65-75° C. or such as 65-75° C.

The enzymatic hydrolysis of the phospholipids may have a duration of 6 hours or less, such as 4 hours or less, such as a duration of 0.5-6 hours, or 0.5-4 hours, or such as a duration of 5 minutes-4 hours, such as 5 minutes to 2 hours, 5 minutes to 1 hour or such as 5-30 minutes.

The one or more enzymes having phospholipid degrading activity may be dosed in a total amount corresponding to 0.1-30 mg enzyme protein.

In the method according to the invention, the vegetable oil is preferably contacted with one or more phospholipid degrading enzymes under conditions such that the number of intact phospholipid molecules is reduced by 30-100%, such as by 30-90%, 30-80%, 30-70% or such as by 30-60% during the enzymatic hydrolysis. The percentage of intact phospholipid molecules may be the determined by the percentage of phosphatidylcholine (PC)+, phosphatidylethanoamine (PE)+phosphatidylinositol (PI))+phosphatidic acid (PA (PC+PE+PI+PA) present after the reaction relatively to the content of PC+PE+PI+PA in the oil before the reaction. The content of the phospholipids can be determined by 31P-NMR analysis or by Liquid chromatography—mass spectrometry (LC-MS). In some embodiments, the vegetable oil is contacted with one or more phospholipid degrading enzymes under conditions such that the enzyme reaction results in at least 10% reduction in the content of PC+PE+PI+PA in the oil, such as at least 25%, or at least 40% reduction in the content of PC+PE+PI+PA in the oil. It is to be understood that the benefit in terms of increased yield provided by the process of the invention will provide does not require a complete or near-complete hydrolysis of the Phosholipids; even a partial hydrolysis of the phospholipids present in the oil will improve the oil yield and the ease of separation of the soap phase after chemical refining.

It is preferred that the vegetable oil, when having been subject to chemical refining according to the method of the invention, contains phospholipids in amounts corresponding to 20 ppm Phosphorous or less, such as 15 ppm or less, such as 10 ppm or less, or such as 5 ppm or less. Preferably, the amounts of phospholipids are determined according to AOCS Official Method Ca 20-99 (2009), Analysis for Phosphorous in Oil by Inductively Coupled Plasma Optical Emission Spectroscopy (ICP-OES), Official Methods and Recommended Practices of the AOCS, AOCS Press, Champaign IL. Further guidance on how to determine the amounts of phosphorous in oil is provided in Z. Benzo et al.: Determination of phosphorus in edible oils by inductively coupled plasma—Atomic emission spectrometry and oil-in-water emulsion of sample introduction, Journal of the American Oil Chemists' Society, September 2000, Volume 77, Issue 9, pp 997-1000.

In further preferred embodiments of the invention, the vegetable oil and said one or more enzymes having phospholipid degrading activity are incubated for 0.1-6 hours, such as for example 0.25-6 hours, or for example 0.5-6 hours under a set of conditions comprising
a) A temperature in the range of 45-90° C. or such as in the range of 60-80° C.;
b) A pH in the range of about 1.5 to about 12.0, such as in the range of 1.5 to 7.0, in the range of pH 4-7, in the range of 3-6, in the range of 6-9, or in the range of pH 7-12.
c) Agitation or mixing, such as by shear mixing, high shear mixing, cavitation mixing or ultrasound.

In preferred embodiments of the invention, the chemical refining is performed subsequent to the enzymatic hydrolysis. In further preferred embodiments, the chemical refining is performed immediately after enzymatic hydrolysis, with no intermediate steps of separation. As mentioned above, the chemical refining step may be performed in the same reaction vessel as the enzymatic hydrolysis was performed in.

The chemical refining when performed according to the invention, may comprise providing an admixture of the vegetable oil with alkali, such as an admixture of the reacted mixture of said vegetable oil as defined above, with alkali.

The alkali is preferably dosed in amounts, which are more than stoichiometric amounts relative to the amounts of free fatty acids present in the oil. As the skilled person will understand in the context of the present disclosure, the amount of alkali dosed in the process is preferably more than the amount, which is sufficient to neutralize free fatty acids, and any chelating agent, such as citric, lactic or phosphoric acid.

In particular, the alkali may be selected from NaOH, KOH, sodium carbonate and combinations thereof.

The inventors have shown that surprisingly, the relationship between the amounts of alkali used to neutralize any acid pre-treatment, and the amount of alkali dosed in the chemical refining, can have a beneficial effect on the phosphor reduction and FFA acid content in the final sample (see Example 12). Accordingly, particular embodiments of the invention relate to methods according to the invention wherein the amount of alkali added in the chemical refining constitutes at least 60% of the total amount of alkali added in the method (i.e., the alkali added after acid pre-treatment in order to adjust pH prior to enzymatic hydrolysis, together with the alkali added for the chemical refining); preferably said amount of alkali added in alkaline refining step is in the range from 60-90%, such as from 60-85%, 60-80%, 60 to 78%, or for example from 62 to 76%.

Alternatively, the invention may be described as wherein the amount of alkali (e.g. NaOH) added in pH adjustment step after acid pre-treatment constitutes at most 40% of the total amount of alkali (i.e., the alkali added after acid pre-treatment in order to adjust pH prior to enzymatic hydrolysis, together with the alkali added for the chemical refining); preferably said amount of base added in pH adjustment step is in the range from 10-40%, such as from 15-40%, 20-40%, such as 40-22%, or for example from 24% to 48%.

In the process according to the invention, the admixture of said vegetable oil and said alkali is preferably incubated from 1 minute to 8 hours, such as from 1 minute to 5 hours, from 1 minute to 2 hours, from 5 minutes to 8 hours, from 5 minutes to 5 hours, from 5 minutes to 2 hours, from 10 minutes to 5 hours, from 10 minutes to 2 hours, from 20 minutes to 5 hours or from 20 minutes to 2 hours.

The inventors have surprisingly shown that introduction of a further acidification step, performed after enzymatic hydrolysis and prior to chemical refining can reduce the amount of phosphor in the final sample after degumming (see Example 14). Thus, some embodiments relate to the method according to the invention, further comprising a step of acidification, performed after enzymatic hydrolysis and prior to chemical refining.

Particular embodiments of the invention relate to where the amount of equivalents of base used to neutralize the acid of the pretreatment is in the range of from 0.5 to 7 equivalents, such as 0.5 to 6, 0.5 to 5, or such as 1.2 to 7 equivalents, such as from 1.5 to 6, 1.5 to 5 equivalents; or for example 2 to 7, 3 to 7 or such as 3 to 7 or 3 to 5 equivalents to the acid, and the method comprises a further acidification step as described.

In particular embodiments of the invention comprising the further acidification step, the one or more phospholipid degrading enzymes comprise or consist of SEQ ID NO. 11 and SEQ ID NO. 13.

The chemical refining as performed according to the invention preferably comprises separating gums and/or soapstock from oil.

Accordingly, the method of the invention may comprise transferring the admixture of vegetable oil and a chemical, such as the admixture of the reacted mixture of said vegetable oil and a chemical to a separator, preferably a centrifugal separator or a horizontal settler.

In the industry, chemical reefing is generally performed using the so-called “Long-Mix” or “Short-Mix” processes or variations thereof. In the “Long Mix process”, a relatively large excess of caustic is mixed into the oil at a relatively low temperature (e.g. 20-40° C.), a holding time with agitation of 3-6 minutes being introduced whereupon the oil/soap mixture is broken by heating it to 60-80° C. The mixture is then fed to a separator; e.g. a centrifugal separator, and the oil stream leaving the centrifuge is heated, water washed and dried; e.g. in a vacuum spray drier. In the “Short-Mix process”, relatively small excess of alkali is added to the oil, whereupon the mixture is fed almost immediately to separator; e.g. a centrifugal separator, water washed and dried.

In both processes the oil may subsequently be bleached to remove color compounds and deodorized to remove volatile odor and flavor compounds.

Hence, in particular embodiments, the method according to the invention comprises
i) Admixing the vegetable oil or the reacted mixture as defined in herein above, with alkali at a temperature of 20-90° C., for example 20-80° C., such as 20-40° C., the amounts of alkali being more than stoichiometric amounts,
ii) Incubating the admixture of vegetable oil and alkali or admixture of reacted mixture and alkali at a temperature of 20-80° C., such as of 20-40° C., for 2-15 minutes with agitation;
iii) Increasing the temperature of the admixture to 55-95° C., such as to 55-85° C.; and
iv) When a temperature of 55-95° C., such as 55-85° C., has been reached, feeding the admixture to a separator to separate gums and/or soapstock from oil.

In alternative embodiments of the invention, the method comprises
i) Admixing the vegetable oil or the reacted mixture as defined above with alkali, the amounts of alkali being more than stoichiometric amounts.
ii) Feeding the admixture of vegetable oil, acid and alkali or the admixture of reacted mixture, acid and alkali to a separator to separate gums and/or soapstock from oil.

The “Short-Mix” and “Long-Mix” processes for caustic refining are disclosed e.g. in: A. J. Dijkstra: Degumming, Refining, Washing and Drying Fats and Oils; in Proceedings of the World Conference on Oilseed Technology and Utilization (1992), Budapest, Hungary; T. H. Applewhite (ed); pp. 138-151; and in: Lipid Handbook, 31d Edition; F. D. Gunstone, J. L. Harwood, A. J. Dijkstra (Eds.), CRC Press, Taylor & Francis Group, 6000 Broken Sound Parkway NW, Suite 300, Boca Raton, FL 33487-2742, © 2007 by Taylor & Francis Group, LLC; see chapter 3: Production and refining of oils and fats, A. J. Dijkstra and J. C. Segers: Long-Mix process is described on age 193, Short-Mix process is described on page 195.

Caustic refining using pressurized equipment using the Nano Neutralization Process is disclosed at www.nanoneutralization.com.

The said one or more enzymes having phospholipid degrading activity may comprise an enzyme having phospholipase A activity, an enzyme having phospholipase C activity, a lyso-phospholipase or a mixture thereof.

Several types of phospholipases are known which differ in their specificity according to the position of the bond attacked in the phospholipid molecule. Phospholipase A1 (PLA1) removes the 1-position fatty acid to produce free fatty acid and 1-lyso-2-acylphospholipid. Phospholipase A2 (PLA2) removes the 2-position fatty acid to produce free fatty acid and 1-acyl-2-lysophospholipid. The term phospholipase B (PLB) is used for phospholipases having both A1 and A2 activity. Phospholipase C (PLC) removes the phosphate moiety to produce 1,2 diacylglycerol and phosphate ester. Phospholipase D (PLD) produces 1,2-diacylglycero-phosphate and base group (See FIG. 1).

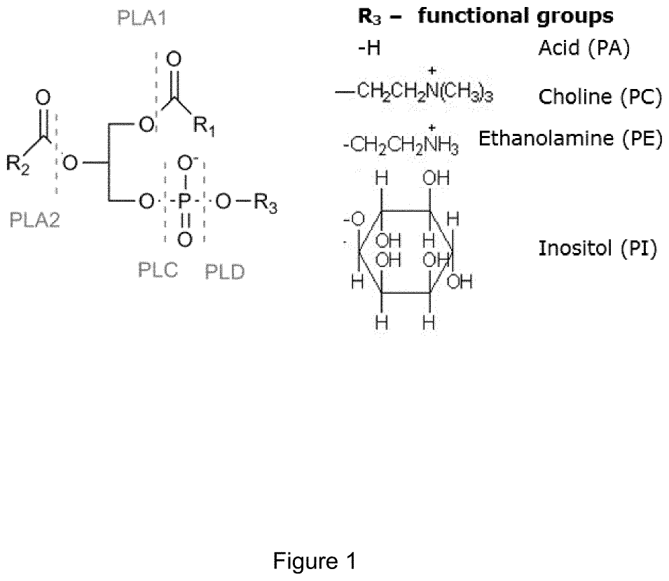

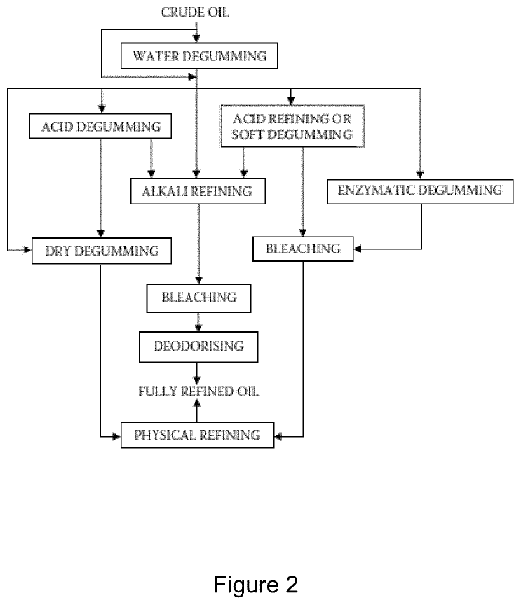

For a review on enzymatic degumming see Dijkstra 2010 Eur. J. Lipid Sci. Technol. 112, 1178. The use of Phospholipase A and/or phospholipase C in degumming is for example described in Clausen 2001 Eur J Lipid Sci Techno 103 333-340, WO 2003/089620 and WO 2008/094847. Phospholipase A solutions generate lysophospholipid and free fatty acids resulting in oil loss. Phospholipase C on the other hand has the advantage that it produces diglyceride (FIG. 2) which will remain in the oil and therefore will reduce losses. There are four major phospholipids in vegetable oil phosphatidylcholine (PC), phosphatidylethanolamine (PE), phosphatidic acid (PA) and phosphatidyl inositol (PI). Phospholipase C enzymes have different specificity towards these phospholipids. A commercially available phospholipase C is Purifine of Verenium/DSM (Dijkstra, 101st AOCS Annual Meeting 10. May 2010) which has specificity towards PC and PE. WO07/059927 describes a thermostable Bacillus PLC for degumming. WO 2012/062817 describes a fungal PLC with specificity towards all four phospholipids.

For the purpose of the present invention it may be preferred that the enzyme or enzymes having phospholipase degrading activity includes a PLC: Besides the yield increase observed by the inventors, it is also of relevance that hydrolysis of phospholipids by PLC does not lead to formation of free fatty acids. In general, it is desired that the production of free fatty acids is minimized during processing of vegetable oil.

In the context of the present invention it has been observed that the yield gain is generally lower when using a PLA to hydrolyse the phospholipids prior to the chemical refining, as compared to the use of PLC. However, additional benefits of the process according to the present invention, which includes lower levels of phospholipids in the resulting soapstock and a lower viscosity of the soapstock, are also achieved with the use of a PLA. In certain embodiments of the invention a lysophospholipase may be preferred as it converts emulsifying Lyso-phospholipids into non-emulsifying compounds.

In relation to the method of the invention, the one or more phospholipid degrading enzymes may have one or more of the following properties:
i) A dissociation temperature (Td) in the range of 50-95° C., e.g. 60-95° C., 70-95° C. such as in the range of 70-90° C.;
ii) A pH optimum in the range pH 3-12, such as in the range of pH 4-7, or such as a pH in the range of 3-6, e.g. 3.5-6 or 4-6, or such as a pH in the range of 5-9, e.g. 6-9 or 6-8, or such as in the range of pH 7-12, e.g. 8-12 or 8-10.

Preferably, the one or more phospholipid degrading enzymes has/have a reaction rate towards the phospholipids in a vegetable oil to which one or more chelation agents capable of complexing Ca and/or Mg ions have been added, said reaction rate being at least 30%, such as at least 40%, at least 50%, at least 60%, at least 70% at least 80% or such as at least 90% of the reaction rate of the one or more phospholipid degrading enzymes towards the phospholipids in said vegetable oil to which no chelation agent(s) have been added. As set forth above, suitable chelation agents may be selected from the group consisting of citric acid, phosphoric acid, lactic acid and EDTA. In these embodiments, the vegetable oil is preferably crude soybean oil and the chelating agent is preferably citric acid, added in amounts corresponding to 500-1000 ppm, such as 650 ppm.

The one or more phospholipid degrading enzymes may in particular be selected from the group consisting of:
a. A phospholipase C having specificity for Phosphatidylinositol (PI),
b. A phospholipase C having specificity for phosphatidyl choline (PC) and Phosphatidyl ethanolamine (PE), preferably Bacillus macauensis PLC, SEQ ID NO. 9
c. A phospholipase C having specificity for Phosphatidyl choline (PC), Phosphatidyl ethanolamine (PE) Phosphatidic acid (PA) and Phosphatidylinositol (PI),
d. A combination of a phospholipase A and a phospholipase C, such as a phospholipase C as defined in a) or b),
e. A combination of a phospholipase A and a lyso-phospholipase.
f. A phospholipase A,
g. A combination of a) and b) or combinations thereof.

In specific embodiments of the invention, the phospholipase A is selected from the group consisting of:
a. A polypeptide having at least 60% sequence identity, such as at least 75% sequence identity, at least 80%, at least 85%, at least 90%, at least 91%, at least 92%, at least 93%, at least 94%, at least 95%, at least 96%, at least 97%, at least 98%, at least 99% or at least 100% sequence identity to the mature polypeptide of any one of SEQ ID NOs: 1, 4 and 7
b. A polypeptide having at least 60% sequence identity, such as at least 75% sequence identity, at least 80%, at least 85%, at least 90%, at least 91%, at least 92%, at least 93%, at least 94%, at least 95%, at least 96%, at least 97%, at least 98%, at least 99% or 100% sequence identity to the polypeptide set forth in any one of SEQ ID NOs: 2, 5 and 8;
c. A fragment of the polypeptide of (a) or (b), that has phospholipase A activity.

In further specific embodiments of the invention the phospholipase A is selected from the group of commercially available PLAs, including PLA Lecitase® 10 L, Lecitase® Novo, Lecitase® Ultra and Quara® LowP, all available from Novozymes A/S, and GumZyme™ available from DSM, LysoMax® Oil available from DuPont, and ROHALASE® PL-XTRA and ROHALASE® mpl available from AB Enzymes.

Preferably, the polypeptides of the present invention have at least 20%, e.g., at least 40%, at least 50%, at least 60%, at least 70%, at least 80%, at least 90%, at least 95%, or at least 100% of the phospholipase A activity of the mature polypeptide of SEQ ID NO: 1 and/or of the polypeptide of SEQ ID NO: 3.

In particular embodiments according to the invention, one of said one or more phospholipid degrading enzymes is a variant of the mature polypeptide mature polypeptide of any one of SEQ ID NOs: 1, 4 and 7, or is a variant of the polypeptide set forth in any one of SEQ ID NOs: 2, 5 and 8, comprising a substitution, deletion, and/or insertion at one or more positions. In particular the said variant may comprise a substitution, deletion, and/or insertion at no more than 20 positions, such as at no more than 19, 18, 17, 16, 15, 14, 13, 12, 10, 9, 8, 7, 6, 5, 4, 3, 2 or 1 position(s).

In other embodiments, one of said one or more phospholipid degrading enzymes comprises, consists essentially of, or consists of the sequence set forth in any one of SEQ ID NOs: 2, 5 and 8. In the method according to the invention, the said phospholipase C may be selected from the group consisting of:
a. A polypeptide having at least 60% sequence identity, such as at least 75% sequence identity, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of any one of SEQ ID NOs: 9 (Bacillus macauensis PLC), 11 (Bacillus thuringiensis PLC), 13 (Pseudomonas sp. PI specific PLC), 15 (P. emersonii PLC); 17 (Kionochaeta PLC), 19 (N. mariannaeae PLC), 22 (Rasamsonia PLC), 25 (T. Spiralis PLC), 28 (T. harzianum PLC),
b. A polypeptide having at least 60% sequence identity, such as at least 75% sequence identity, at least 80%, at least 85%, at least 90%, at least 91%, at least 92%, at least 93%, at least 94%, at least 95%, at least 96%, at least 97%, at least 98%, at least 99% or 100% sequence identity to the polypeptide set forth in any one of SEQ ID NOs: 10, 12, 14, 16, 18, 20, 23, 26, 29; and
c. A fragment of the polypeptide of (a) or (b) that has phospholipase C activity.

In particular, the one of said one or more phospholipid degrading enzymes may be a variant of the mature polypeptide mature polypeptide of any one of SEQ ID NOs: 9, 11, 13, 15, 17, 19, 22, 25 and 28, or is a variant of the polypeptide set forth in any one of SEQ ID NOs: 10, 12, 14, 16, 18, 20, 23, 26 and 29, comprising a substitution, deletion, and/or insertion at one or more positions.

In other embodiments, the one of said one or more phospholipid degrading enzymes comprises, consists essentially of, or consists of the sequence set forth in any one of SEQ ID NOs: 10, 12, 14, 16, 18, 20, 23, 26 and 29.

In further specific embodiments of the invention the phospholipase C is selected from the group of commercially available PLCs, including Purifine® available from DSM and Quara® Boost, available from Novozymes A/S.

One preferred embodiment relates to wherein said at least one phospholipid degrading enzyme comprises or consists of SEQ ID NO. 9, (Bacillus macauensis PLC).

Further preferred embodiments relate to wherein said at least one phospholipid degrading enzyme comprises or consists of a phospholipase C having specificity for Phosphatidylinositol (PI), and a phospholipase C having specificity for phosphatidyl choline (PC) and Phosphatidyl ethanolamine (PE), preferably Bacillus macauensis PLC, SEQ ID NO. 9.

The lysophospholipase may be selected from the group of commercially available lysophospholipases, including Finizym™, which is available from Novozymes.

The process of the invention may further include steps of bleaching, deodorization, and fractionation.

The usual method of bleaching is by adsorption of the color producing substances on an adsorbent material. Acid-activated bleaching earth or clay, sometimes called bentonite, is the adsorbent material that has been used most extensively. This substance consists primarily of hydrated aluminum silicate. Anhydrous silica gel and activated carbon also are used as bleaching adsorbents to a limited extent.

Deodorization of fats and oils is removal of the relatively volatile components from the fat or oil using steam. This is feasible because of the great differences in volatility between the substances that give flavors, colors and odors to fats and oils and the triglycerides. Deodorization is carried out under vacuum to facilitate the removal of the volatile substances, to avoid undue hydrolysis of the fat, and to make the most efficient use of the steam. In the case of vegetable oils, sufficient tocopherols remain in the finished oils after deodorization to provide stability.

Deodorization does not have any significant effect upon the fatty acid composition of most fats or oils. Depending upon the degree of unsaturation of the oil being deodorized, small amounts of trans fatty acids may be formed by isomerization.

Fats that are solid at room temperature usually contain a mixture of many individual triglycerides, all of which have different melting points. These components can be separated from one another by the fractionation process.

The result of fractionation is the production of two components, called fractions that typically differ significantly from each other in their physical properties. The fractions can be fractionated again (“double” fractionation) to produce additional fractions, which will have unique physical properties. The process was originally developed to fractionate animal fats such as beef tallow.

There are two types of fractionation techniques: dry and wet. Dry fractionation refers to a process that does not use a solvent to assist in the separation of the fat components. The fat is first melted, and then cooled slowly to generate large, high melting point fat crystals. The slurry of crystals suspended in liquid oil is transferred to a high-pressure filter press where the liquid (olein) fraction is squeezed out and the hard (stearin) fat is retained on the filter. This process is widely applied to palm oil and palm kernel oil to generate several unique products from a single natural source, without the need for chemical processing. Fractions produced in this way can be blended together or mixed with liquid vegetable oils to make a wide variety of functional products for many food applications.

A second aspect of the invention provides the use of a phospholipid degrading enzyme to hydrolyze phospholipids in a vegetable oil, wherein the vegetable oil is contacted with the phospholipid degrading enzyme, and thereafter subjected to chemical refining.

When using the phospholipid degrading enzyme according to the invention, it is preferred that
enzymatic hydrolysis of said phospholipids is performed in a reaction mixture comprising a heavy phase or aqueous phase and a light phase or oil phase/hydrophobic phase, and
there is no reduction or no substantial reduction of the heavy phase volume or separation of gums/heavy phase from oil before said chemical refining.

In a third aspect, the invention provides a refined vegetable oil, a separated gum fraction or a soapstock, which is obtainable or is obtained by a method as defined in the first aspect of the invention. In particular embodiments, the oil according to the invention contains an amount of diglycerides of 0.1% (w/w), such as 0.2% (w/w) or more, or such as 0.3% (w/w) or more. The soapstock may have a lower viscosity and may a lower content of phospholipids than soapstock from a conventional chemical refining process.

In a fourth aspect, the invention provides an isolated or purified polypeptide having phospholipase A activity, selected from the group consisting of:
a. A polypeptide having at least 75% sequence identity, such as at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of any one of SEQ ID NOs: 4 and 7,
b. A polypeptide having at least 75% sequence identity, such as at least 80%, at least 85%, at least 90%, at least 91%, at least 92%, at least 93%, at least 94%, at least 95%, at least 96%, at least 97%, at least 98%, at least 99% or 100% sequence identity to the polypeptide set forth in any one of SEQ ID NOs: 5 and 8;
c. A fragment of the polypeptide of (a) or (b), that has phospholipase A activity.

In certain embodiments, the polypeptide is a variant of the mature polypeptide mature polypeptide of any one of SEQ ID NOs: 4 and 7, or may be a variant of the polypeptide set forth in any one of SEQ ID NOs: 5 and 8, comprising a substitution, deletion, and/or insertion at one or more positions. In particular the said variant may comprise a substitution, deletion, and/or insertion at no more than 20 positions, such as at no more than 19, 18, 17, 16, 15, 14, 13, 12, 10, 9, 8, 7, 6, 5, 4, 3, 2 or 1 position(s).

In particular, the polypeptide may comprise, consist essentially of, or consist of the sequence set forth in SEQ ID NO: 5 or SEQ ID NO: 8.

In a fifth aspect, the invention provides an isolated or purified polypeptide having phospholipase C activity, selected from the group consisting of:
a. A polypeptide having at least 60% sequence identity to the mature polypeptide of any one of SEQ ID NOs: 22, 25 and 28,
b. A polypeptide having at least 60% sequence identity to the polypeptide set forth in any one of SEQ ID NOs: 23, 26 and 29; and
c. A fragment of the polypeptide of (a) or (b) that has phospholipase C activity.

In some embodiments of the invention, the polypeptide is a variant of the mature polypeptide mature polypeptide of any one of SEQ ID NOs: 22, 25 and 28, or is a variant of the polypeptide set forth in any one of SEQ ID NOs: 23, 26 and 29, comprising a substitution, deletion, and/or insertion at one or more positions. In particular, the said variant may comprise a substitution, deletion, and/or insertion at no more than 20 positions, such as at no more than 19, 18, 17, 16, 15, 14, 13, 12, 10, 9, 8, 7, 6, 5, 4, 3, 2 or 1 position(s).

The polypeptide may comprise, consist essentially of, or consist of the sequence set forth in SEQ ID NO: 23, 26 or 29.

In a sixth aspect, the invention comprises a composition comprising the polypeptide according to the fourth or fifth aspect of the invention as set forth above.

In a seventh aspect, the invention provides an isolated or purified polynucleotide encoding the polypeptide according to the fourth or fifth aspect of the invention as set forth above.

The sequence of a polynucleotide encoding the polypeptide set forth in SEQ ID NO: 4 or 5 is set forth in SEQ ID NO: 3. The sequence of a polynucleotide encoding the polypeptide set forth in SEQ ID NO: 7 or 8 is set forth in SEQ ID NO: 6.

The sequence of a polynucleotide encoding the polypeptide set forth in SEQ ID NO: 22 or 23 is set forth in SEQ ID NO: 21. The sequence of a polynucleotide encoding the polypeptide set forth in SEQ ID NO: 25 or 26 is set forth in SEQ ID NO: 24. The sequence of a polynucleotide encoding the polypeptide set forth in SEQ ID NO: 28 or 29 is set forth in SEQ ID NO: 27

In an eight aspect, the invention provides nucleic acid construct or expression vector comprising a polynucleotide as provided in the seventh aspect of the invention, wherein the polynucleotide is preferably operably linked to one or more control sequences that direct the production of the polypeptide in an expression host.

In a ninth aspect, the invention provides a recombinant host cell comprising the polynucleotide as provided in the seventh aspect of the invention, operably linked to one or more control sequences that direct(s) the production of the polypeptide.

In particular, the invention pertains to a host cell, wherein the polypeptide is heterologous to the recombinant host cell.

The recombinant host cell may be one, wherein at least one of the one or more control sequences is heterologous to the polynucleotide encoding the polypeptide.

In a tenth aspect, the invention provides a method of producing the polypeptide according to the fourth or fifth aspect of the invention, comprising cultivating a cell, which in its wild-type form produces the polypeptide, under conditions conducive for production of the polypeptide.

The method may further comprise a step of recovering the polypeptide.

The eleventh aspect of the invention pertains to a method of producing a polypeptide having phospholipase A activity or a polypeptide having phospholipase C activity, comprising cultivating the recombinant host cell according to the ninth aspect of the invention under conditions conducive for production of the polypeptide.

The method may further comprise a step of recovering the polypeptide.

### EXAMPLES

### Example 1

Enzyme: Bacillus macauensis PLC: Mature polypeptide of SEQ ID NO: 9

Oil: Crude soy bean oil. Content of the individual phospholipid components is measured by the amount of phopshorous (P) from the components as ppm P.

| PC | PI | PE | PA |
|---|---|---|---|
| 433 ppm P | 231 ppm P | 327 ppm P | 90 ppm P |

Enzyme Reaction and Alkaline Refining Assay

Performance of the enzyme was tested in an enzyme reaction assay that mimics industrial scale conditions.

Crude soybean oil (75 g) was initially acid pretreated by addition of 650 ppm citric acid. In test samples, the pH was raised to ˜6 by addition of 1.5 eqv. NaOH; control samples (blank; no enzyme), the pH was maintained at ˜4. Samples were subject to mixing in ultrasonic bath (BRANSON 5800) for 5 min and incubation in rotator for 15 min at 70° C. The enzyme reaction was conducted in low aqueous system (2% water total based on oil amount) in 100 ml centrifuge tubes, cylindrical, conical bottom. Samples were ultrasonic treated for 5 min, followed by incubation in rotator in a heated cabinet at 70° C. with stirring at 20 rpm for 1 hour. After the enzyme reaction a high amount of alkaline was added to the solution by the following procedure:
i) 1141 ppm NaOH was added to the oil;
ii) The reactor tubes were hand shaken for 10 s and 5 min in the ultrasonic bath;
iii) The reaction was left for 0.5 h at 70° C. at 20 rpm;
iv) After reaction, 10 mL samples were centrifuged in Hot spin centrifuge; at 700 g at 85° C. for 5 min (Koehler Instruments, K600X2 oil centrifuge).

Samples and Conditions:

TABLE 1
Table 1: Sample condition; pH 4 and 6 at
70° C. for 1 hour for enzymatic reaction

| | | | Acid/base |
|---|---|---|---|
| | | Enzyme | conditions for enzyme |
| Flask | Label | concentration | reaction |
| 1 | Blank | — | 650 ppm citric acid~pH |
| 2 | Blank | — | 4 |
| 3 | B. macauensis PLC | 2 mg EP/kg oil | 650 ppm citric acid + |
| 4 | B. macauensis PLC | | 1.5 eqv. NaOH~pH 6 |
| 5 | B. macauensis PLC | 4 mg EP/kg oil | |
| 6 | B. macauensis PLC | | |

Yield Estimate by Gums Volume:

Gum volume was by visual reading using the glass tube scale in ASTM D91 centrifugation tubes and the gum volumes were then used to calculate the oil yield.

The dry matter content of the gums was taken into consideration when calculating the oil yield in order for accuracy purposes: The gums volumes can have the same value in an enzyme treated and a blank sample, but the dry matter contents are significantly different. The relative oil content is thus calculated using the following equations:

$$Yield = \frac{{Oil}_{l} - Gums \times \frac{Dry matter \%}{100 \%}}{{Oil}_{l}} \times 100 \%$$

Oil: Initial volume of the crude oil before degumming (mL)
Gums: Measured gum volume (mL)
Dry matter: The dry matter content in the gums (%)
Yield: The yield is an estimate of the recovered oil volume (%)

Diglyceride Content:

Diglyceride content was determined by High-performance liquid chromatography (HPLC) coupled to Charged Aerosol Detector (Corona Veo) according to the principles described in AOCS Official Method Cd 11d-96. DIONEX equipment and Kinetex 2.6u HILIC 100A, 150×4.6 mm, Phenomenex column was applied.

Quantification of Phospholipids

Phospholipids in oil were determined by 31P NMR quantification using the following procedure: To the oil sample was added 0.500 mL internal standard (IS) solution, followed by 0.5 mL CDCl₃ and 0.5 mL Cs-EDTA buffer. The sample was shaked for 5 min, and then centrifuged (tabletop centrifuge, 5 min, 13,400 rpm) to get phase separation. The lower phase was transferred to a NMR-tube. P-NMR was performed with 128 scans and a delay time of 5 s. All signals were integrated. Assignments (approx. ppm): 1.5 (PA), −0.1 (PE), −0.6 (PI), −0.8 (PC). The concentration of each species was calculated as “ppm P”, i.e. mg elemental P per kg oil sample. Hence, ppm P=I/I(IS)*n(IS)*M(P)/m(oil). Residual phospholipid content was calculated as the ratio of enzyme treated sample vs blank. The internal standard solution is 2 mg/mL triphenylphosphate in methanol. The Cs-EDTA buffer was prepared as follows: EDTA (17.55 g) was dispersed in water (approx. 20 mL). The pH was adjusted to 7.5 using 50% w/w CsOH. This gave a clear solution. Water was added up to 100 mL to give a concentration of 0.6 M EDTA.

Total Phosphorous:

Total phosphorus was measured by Inductively coupled plasma optical emission spectrometry (ICP-OES) with an accuracy of approximately ±1 ppm P.

Results:

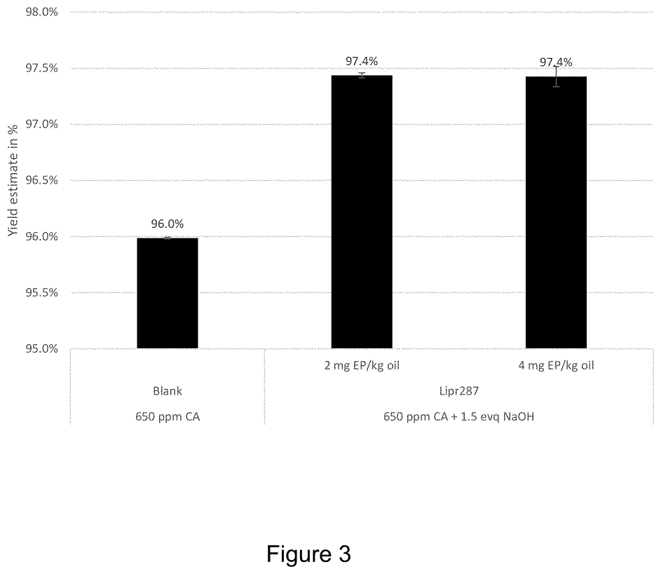

The yield was measured by gums level and the dry matter content of the gums. There was a clear and significant benefit when using B. macauensis PLC before alkaline degumming. The yield is the same for both enzyme concentrations. The estimated gain is 1.4% more oil than blank. Results are shown in FIG. 3.

The di-glyceride content was measured over time to follow the enzyme activity of PLC. The blank has, as expected, no increase of the di-glyceride content. The B. macauensis PLC has significant di-glyceride increased content compared to the blank and crude oil, 1.08-1.33% more DG. The two enzyme dosages have more or less the same increase of di-glyceride content.

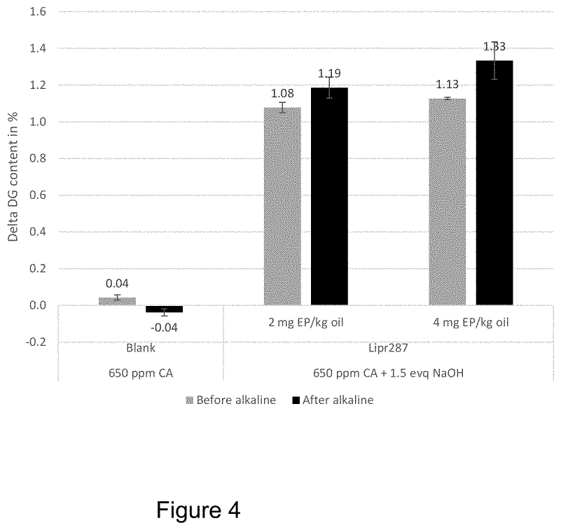

A small increase in the di-glyceride level after the alkaline stage was observed. The 0.1 to 0,2% extra di-glycerides potentially formed during the alkaline stage are not alarmingly high. Results are shown in FIG. 4.

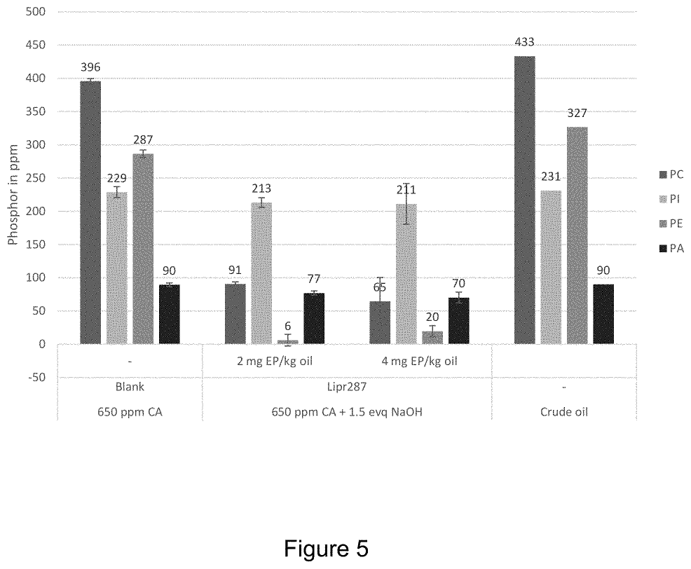

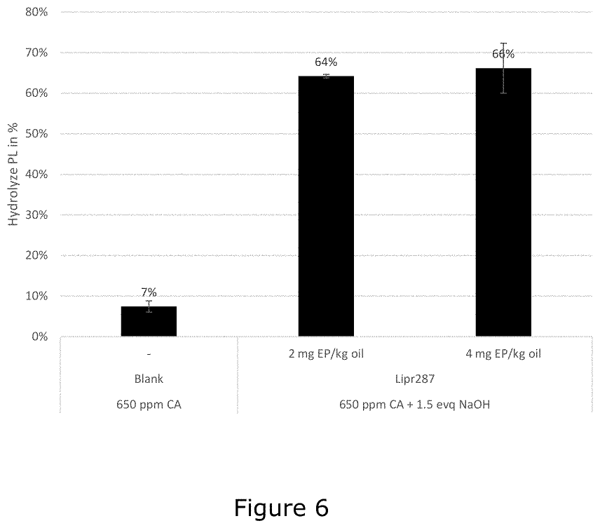

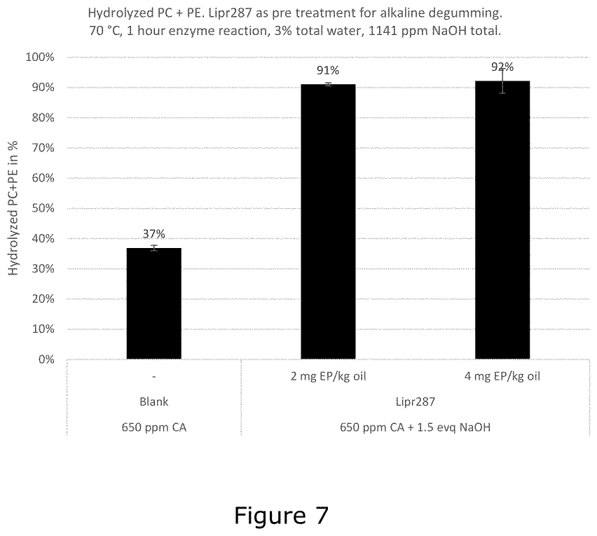

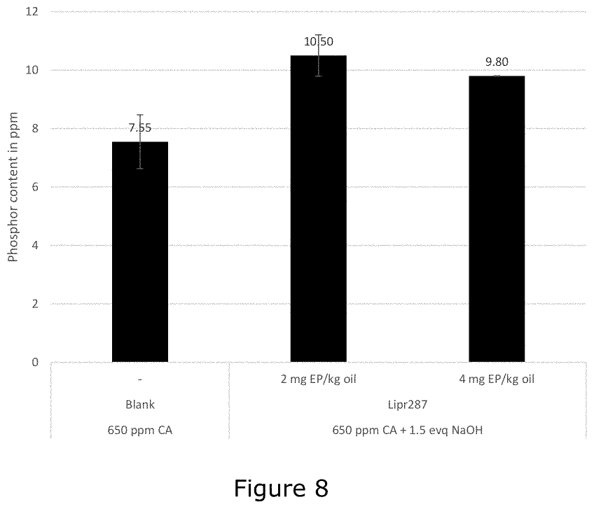

The intact phospholipids were measured to follow the conversion of phospholipids. B. macauensis PLC converts 66% of all four phospholipids after 1-hour reaction time. B. macauensis PLC is specific for PC and PE and for these two phospholipid species the hydrolysis reach 92%. Results are shown in FIGS. 5, 6 and 7.

Conclusions:

The trial confirmed the high yield gain (1.4%) delivered by treatment with the B. macauensis PLC. There is no significant different between a 2 and a 4 mg enzyme protein/kg dosage of B. macauensis PLC. The dosage 2 mg EP/kg oil seems to be sufficient or maybe even a high dosage. Only 1-hour enzymatic reaction time gives high hydrolysis of PC and PE (92%). Full degumming observed; residual phosphor in oil is sufficient low

### Example 2

Enzyme: T. leycettanus PLA; mature polypeptide of SEQ ID NO: 1

Bacillus macauensis PLC: Mature polypeptide of SEQ ID NO: 9

Oil: Crude soy bean oil; as disclosed in Example 1

Enzyme Reaction and Alkaline Refining

Performance of the enzymes were tested in an enzyme reaction assay, following the procedure set forth in Example 1. Crude soybean oil (75 g) was initially acid pretreated by addition of 650 ppm citric acid. In samples for testing of Bacillus macauensis PLC, the pH was raised to −6 by addition of 1.5 eqv. NaOH; in control samples (blank; no enzyme) and samples for testing of T. leycettanus PLA, the pH was maintained at ˜4.

After the enzyme reaction, alkaline was added following the procedure described in Example 1.

TABLE 2
Samples and conditions
Table 2: Sample condition; pH 4 and 6 at
70° C. for 2 hours for enzymatic reaction

| | | Enzyme | Acid/base condition |
|---|---|---|---|
| Flask | Label | concentration | for enzyme reaction |
| 1 | Blank | — | 650 ppm citric acid~pH 4 |
| 2 | Blank | — | |
| 3 | T. leycettanus | 30 ppm product | |
| 4 | PLA | | |
| 5 | | | |
| 6 | B. macauensis | 2 mg enzyme | 650 ppm citric acid + 1.5 eqv. |
| 7 | PLC | protein/kg oil | NaOH~pH 6 |
| 8 | | | |

Yield estimate by gum volume and diglyceride content were determined as set forth in Example 1.

Content of free fatty acids was determined according to AOCS Ca 5a-40 Official Method. In brief, a known mass of oil was dissolved in 2-propanol and phenolphthalein was added as indicator. The free fatty acids were then neutralized by titration with a NaOH-solution until occurrence of the first pale permanent pink colour.

Results:

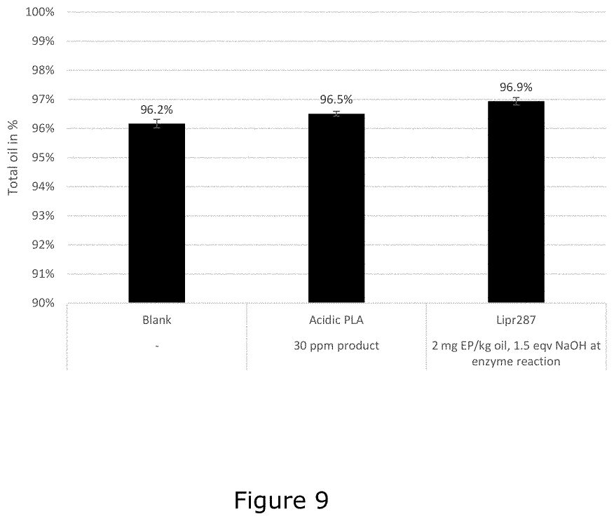

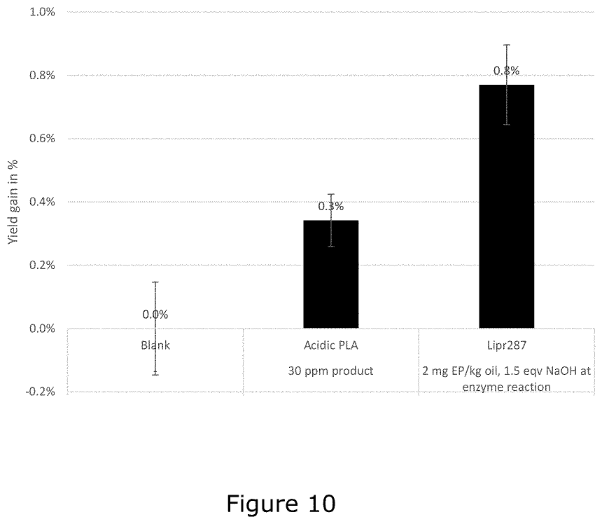

The yield was measured by gums level and the dry matter content of the gums. There is a clear and significant benefit when using 30 ppm T. leycettanus PLA or 2 mg EP/kg oil of B. macauensis PLC before for alkaline degumming. The gain is 0.3% for T. leycettanus PLA and 0.8% for B. macauensis PLC. Results are shown in FIGS. 9 and 10.

The gums phase itself was quite heterogenic after centrifugation and formed hard white crystals. During the dissolving of the gums again with the sodium hydroxide (NaOH) was almost impossible with handshake and Ultra sonic bath. A proper mixing could lead to more yield for the enzymatic treatment.

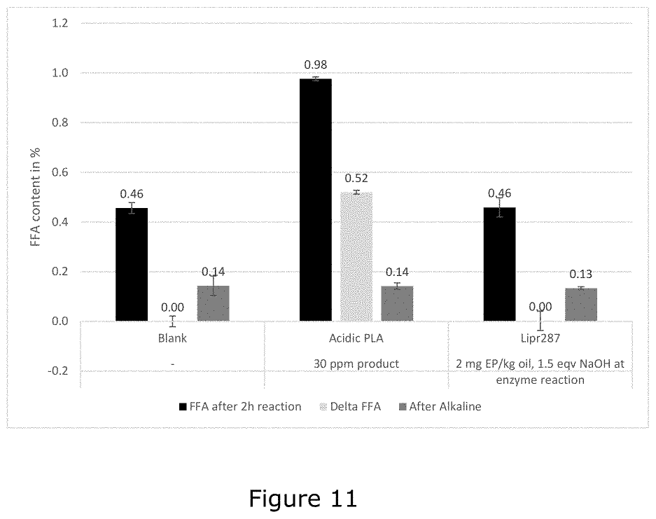

The FFA content was measured over time to follow the enzyme activity of PLA. Results are shown in FIG. 11.

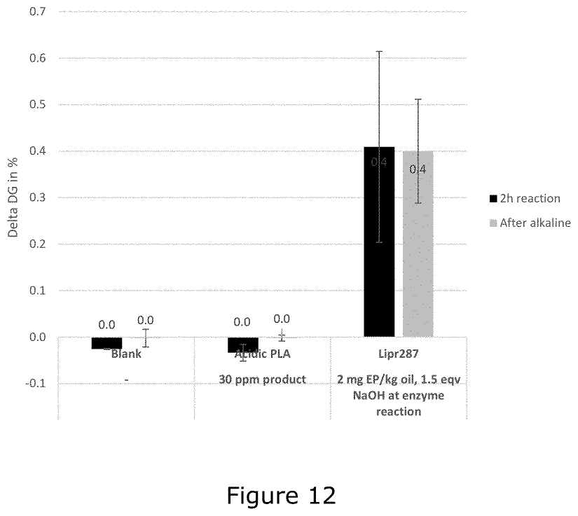

The di-glyceride content was measured over time to follow the enzyme activity of PLC. Results are shown in FIG. 12. As expected, the T. leycettanus PLA and blank has no increase of di-glyceride content. The B. macauensis PLC has significant di-glyceride content compare to the blank. There is high standard variation of the DG content for B. macauensis PLC. The reason could be that the used enzyme tubes had to be defrosted and frozen serval times.

### Example 3: Cryphonectria parasitica PLA and Gloeophyllum trabeum PLA; Hydrolytic Activity and Substrates Specificity as Determined by P-NMR Assay

Origin

Cryphonectria parasitica PLA was cloned from Donor NN008388 Cryphonectria parasitica, from Sweden; 1994.

Gloeophyllum trabeum PLA was cloned from Donor NN050212 Gloeophyllum trabeum from Russia; 1997.

Hydrolytic Activity and Substrates Specificity

Concept

The assay was conducted by incubating the phospholipase with a 10:1 mixture of a crude vegetable oil and aqueous buffer. Enzyme concentration was 10 mg/kg (mg EP per kg oil). The mixture was incubated with vigorous shaking at 50 C for 2 h. The reaction mixture was then analyzed by ³¹P NMR and the amount of remaining (not hydrolyzed, intact) phospholipid quantified. The result is a measure of hydrolytic activity and substrate specificity of the enzyme.

Assay Procedure

The purified enzyme was diluted to 0.09 mg/mL in 100 mM citrate buffer pH 4.0, 5.5 and 7.0. The assay was initiated by adding 25 uL diluted enzyme to 250 uL crude vegetable oil in a 2 mL Eppendorf tube and incubating the mixture in a thermoshaker at 50 C for 2 h. The oil used was a crude soybean oil containing a significant amount of both PA, PE, PI and PC (100-200 ppm P of each).

NMR Analysis

To the oil sample was then added 0.500 mL internal standard (IS) solution, followed by 0.5 mL CDCl₃ and 0.5 mL Cs-EDTA buffer. The sample was shaked for 5 min, and then centrifuged (tabletop centrifuge, 5 min, 13,400 rpm) to get phase separation. The lower phase was transferred to a NMR-tube. ³¹P NMR was performed with 128 scans and a delay time of 5 s. All signals were integrated. Assignments (approx. ppm): 1.5 (PA), −0.1 (PE), −0.6 (PI), −0.8 (PC). The concentration of each species was calculated as “ppm P”, i.e. mg elemental P per kg oil sample. Hence, ppm P=I/I(IS)*n(IS)*M(P)/m(oil). The IS solution is 2 mg/mL triphenylphosphate in MeOH. The Cs-EDTA buffer was prepared as: EDTA (5.85 g) is dispersed in water (approx. 50 mL). The pH was adjusted to 7.5 using 50% w/w CsOH. This gave a clear solution. Water was added up to 100 mL to give a concentration of 0.2 M EDTA.

Results

The results in the tables 14 and 15 below show that both enzymes are active on all four phospholipids and both prefer pH 4 over pH 5.5 and 7.0.

TABLE 14
Phospholipid content (ppm P) of soybean oil
incubated with Gloeophyllum trabeum PLA.

| | | pH | pH | pH |
|---|---|---|---|---|
| | Oil | 4 | 5.5 | 7 |
| PA | 120 | 0 | 112 | 120 |
| PE | 120 | 0 | 112 | 112 |
| PI | 79 | 0 | 87 | 79 |
| PC | 132 | 0 | 141 | 141 |

TABLE 15
Phospholipid content (ppm P) of soybean oil
incubated with Cryphonectria parasitica PLA.

| | | pH | pH | pH |
|---|---|---|---|---|
| | Oil | 4 | 5.5 | 7 |
| PA | 116 | 41 | 112 | 112 |
| PE | 107 | 12 | 12 | 112 |
| PI | 83 | 45 | 79 | 83 |
| PC | 136 | 29 | 132 | 136 |

### Example 16: Trichoderma harzianum PLC; Cloning, Expression, Fermentation and Purification

Genomic DNA was extracted from the strain NN051266 Trichoderma harzianum, using Fast DNA Spin for Soil Kit Cat no. 6560-200 from MP Biochemicals, following the protocol from the supplier.

The D23CR9, P33XXG gene (SEQ ID NO. 27) was amplified by PCR from the genomic DNA. The PCR was composed of 1 μl of genomic DNA of the strain; 2.5 μl of cloning primer forward (SEQ ID NO: 52; and 53) (10 pmol/μl), 2.5 μl of primer cloning primer reverse (SEQ ID NO: 54; and 55) (10 pmol/μl), 25 μl of iProof HF Master Mix (BioRad Cataloge #172-5310), and 19 μl PCR-grade water.

The amplification reaction was performed using a Thermal Cycler programmed for 2 minutes at 98° C. followed by 30 cycles each at 98° C. for 10 seconds and 60° C. for 10 seconds, followed by one cycle at 72° C. for 5 minutes.

P8_43-F
5′ ACACAACTGGGGATCCACCATGCGTCCCAGCTCGACGC-3′
P8_43-F
5′ ACACAACTGGGGATCCACCATGCGTCCCAGCTCGACGC-3′
P8_43-R
5′ AGATCTCGAGAAGCTTAAGCCTTGGCTTTCAACTCATTAGCC 3′
P7_30-R
5′ AGATCTCGAGAAGCTTAAGCCTTGGCTTTCAACTCATTGGC 3′

The country of origin for NN051266 Trichoderma harzianum is China (2008).

4 μl of the PCR product was visualized on a 1.0% agarose gel electrophoresis using TAE buffer. The remaining PCR product was purified using a GFX® PCR DNA and Gel Band Purification Kit (GE Healthcare, HiHerod Denmark) according to manufacturer's instructions. The purified PCR product, corresponding to the NN051266 Trichoderma harzianum PLC gene D23CR9, was cloned into the expression vector pDAu109 (WO 2005/042735) previously linearized with Barn HI and Hind III, using an IN-FUSION™ Dry-Down PCR Cloning Kit (BD Biosciences, Palo Alto, CA, USA) according to the manufacturer's instructions.

A 1 μl volume of the undiluted ligation mixture was used to transform BD Phusion-Blue (Clontech). One colony was selected on a LB agar plate containing 100 μg of ampicillin per ml and cultivated overnight in 2 ml of LB medium supplemented with 100 μg of ampicillin per ml. Plasmid DNA was purified using a Jetquick Plasmid Miniprep Spin Kit (Genomed GmbH, Løhne, Germany) according to the manufacturer's instructions. The NN051266 Trichoderma harzianum PLC gene D23CR9 sequences was verified by Sanger sequencing before heterologous expression. One plasmid designated as P8_43 (containing gene SEQ ID NO: 27) was selected for heterologous expression of the PLC genes in Aspergillus oryzae MT3568 host cells.

Aspergillus oryzae MT3568 strain was used for heterologous expression of the D23CR9, P33XXG gene. A. oryzae MT3568 is an amdS (acetamidase) disrupted gene derivative of Aspergillus oryzae JaL355 (WO 2002/40694) in which pyrG auxotrophy was restored by disrupting the A. oryzae acetamidase (amdS) gene with the pyrG gene. Protoplasts of Aspergillus oryzae MT3568 were prepared according to WO 95/002043.

One hundred μl of Aspergillus oryzae MT3568 protoplasts were mixed with 1-2 μg of the Aspergillus expression vector with the cloned D23CR9 gene and 250 μl of 60% PEG 4000 (Applichem, Darmstadt, Germany) (polyethylene glycol, molecular weight 4,000), 10 mM CaCl₂), and 10 mM Tris-HCl pH 7.5 and gently mixed. After 30 min of incubation at 37° C., 4 ml of topagar (temp. 40° C.) was added, and the protoplasts were spread onto COVE plates for selection. After incubation for 4-7 days at 37° C., spores of four transformants were inoculated into 0.5 ml of DAP-4C-01 medium in 96 deep well plates. After 4-5 days cultivation at 30° C., the culture broths were analyzed by SDS-PAGE to identify the transformants producing the largest amount of recombinant phospholipase C from NN051266 Trichoderma harzianum.

Spores of the best transformant with the NN051266 Trichoderma harzianum PLC gene D23CR9 were spread on COVE plates containing 0.01% TRITON® X-100 in order to isolate single colonies. The spreading was repeated once more before preservation of the clones.

Fermentation for Purification

An Aspergillus oryzae transformant constructed as described above was fermented in 150 ml DAP-4C-01 medium in 500 ml fluted shake flasks incubated at 30° C. in a shaking platform incubator rotating at 150 RPM for 5 days and further used for assays as described below.

### Example 4: Trichurus Spiralis PLC; Cloning, Expression and Fermentation

Genomic DNA was extracted from the strain NN009739 Trichurus spiralis using Fast DNA Spin for Soil Kit Cat no. 6560-200 from MP Biochemicals, following the protocol from the supplier.

The D23YRT, P34CUT gene (SEQ ID NO. 26) was amplified by PCR from the genomic DNA. The PCR was composed of 1 μl of genomic DNA of the strain; 2.5 μl of cloning primer forward (P7_37-F) (10 pmol/μl), 2.5 μl of primer cloning primer reverse (P7_37-R) (10 pmol/μl), 25 μl of iProof HF Master Mix (BioRad Cataloge #172-5310), and 19 μl PCR-grade water.

The amplification reaction was performed using a Thermal Cycler programmed for 2 minutes at 98° C. followed by 30 cycles each at 98° C. for 10 seconds and 60° C. for 10 seconds, followed by one cycle at 72° C. for 5 minutes.

P7_37-F
5′ ACACAACTGGGGATCCACCATGCATCTCACTCGCGTCGC-3′
P7_37-R
5′ AGATCTCGAGAAGCTTAGATTAGGAGTCTCTTGTTCTCCTC
GACC 3′

The country of origin for NN009739 Trichurus spiralis is Denmark (1996).

4 μl of the PCR product was visualized on a 1.0% agarose gel electrophoresis using TAE buffer. The remaining PCR product was purified using a GFX® PCR DNA and Gel Band Purification Kit (GE Healthcare, HiHerod Denmark) according to manufacturer's instructions. The purified PCR product, corresponding to the NN009739 Trichurus spiralis PLC gene D23YRT, was cloned into the expression vector pDAu109 (WO 2005042735) previously linearized with Bam HI and Hind III, using an IN-FUSION™ Dry-Down PCR Cloning Kit (BD Biosciences, Palo Alto, CA, USA) according to the manufacturer's instructions.

A 1 μl volume of the undiluted ligation mixture was used to transform BD Phusion-Blue (Clontech). One colony was selected on a LB agar plate containing 100 μg of ampicillin per ml and cultivated overnight in 2 ml of LB medium supplemented with 100 μg of ampicillin per ml. Plasmid DNA was purified using a Jetquick Plasmid Miniprep Spin Kit (Genomed GmbH, Løhne, Germany) according to the manufacturer's instructions. The NN009739 Trichurus spiralis PLC gene D23YRT sequence was verified by Sanger sequencing before heterologous expression. One plasmid designated as P7_37 (containing gene SEQ ID NO: 14) was selected for heterologous expression of the PLC gene in an Aspergillus oryzae MT3568 host cell.

Aspergillus oryzae MT3568 strain was used for heterologous expression of D23YRT, P34CUT. A. oryzae MT3568 is an amdS (acetamidase) disrupted gene derivative of Aspergillus oryzae JaL355 (WO 2002/40694) in which pyrG auxotrophy was restored by disrupting the A. oryzae acetamidase (amdS) gene with the pyrG gene. Protoplasts of Aspergillus oryzae MT3568 were prepared according to WO 95/002043.

One hundred μl of Aspergillus oryzae MT3568 protoplasts were mixed with 1-2 μg of the Aspergillus expression vector with the cloned D23YRT gene and 250 μl of 60% PEG 4000 (Applichem, Darmstadt, Germany) (polyethylene glycol, molecular weight 4,000), 10 mM CaCl₂, and 10 mM Tris-HCl pH 7.5 and gently mixed. After 30 min of incubation at 37° C., 4 ml of topagar (temp. 40° C.) was added, and the protoplasts were spread onto COVE plates for selection. After incubation for 4-7 days at 37° C., spores of four transformants were inoculated into 0.5 ml of DAP-4C-01 medium in 96 deep well plates. After 4-5 days cultivation at 30° C., the culture broths were analyzed by SDS-PAGE to identify the transformants producing the largest amount of recombinant phospholipase C from NN009739 Trichurus spiralis, and the culture broths were also analyzed in assays for confirmation of activity.

Spores of the best transformant were spread on COVE plates containing 0.01% TRITON® X-100 in order to isolate single colonies. The spreading was repeated once more before preservation of the clone.

Fermentation for Purification

An Aspergillus oryzae transformant constructed as described above was fermented in 150 ml DAP-4C-01 medium in 500 ml fluted shake flasks incubated at 30° C. in a shaking platform incubator rotating at 150 RPM for 5 days and further used for assays as described below.

Medias Used

LB plates were composed of 10 g of Bacto-Tryptone, 5 g of yeast extract, 10 g of sodium chloride, 15 g of Bacto-agar, and deionized water to 1 liter.

LB medium was composed of 10 g of Bacto-Tryptone, 5 g of yeast extract, and 10 g of sodium chloride, and deionized water to 1 liter.
DAP-4C-1
11 g MgSO4,7H2O
1 g KH2PO4
2 g C6H8O7,H2O
20 g Dextrose
10 g Maltose
5.2 g K3PO4,H2O
0.5 g Yeast Extract
0.5 ml KU6 Trace metal sol.(AMG) (MSA-SUB-FS-0042)
Mix until completely solved
1 ml Dowfax 63N10 is added
Adjust volume with Milli-Q-water up to 1000 ml
CaCO3 tabl. a 0.5 g (add 1 tabl./200 ml)
Before inoculation, each shake flask a 150 ml is added 3.5 ml di-Ammoniumhydrogenphosphat (NH4)2HPO4 50%, and 5.0 ml Lactic acid 20%.
KU6 Trace metal sol.(AMG) (MSA-SUB-FS-0042)
6.8 g ZnCl₂
2.5 g CuSO₄·5H₂O
0.13 g Nickel Chloride anhydrous
13.9 g FeSO4·7H₂O
8.45 g MnSO4·H₂O
3 g C₆H₈O₇·H₂O
Ion exchanged water up to 1000 ml

| | Chem. | | 7-cif. | | |
|---|---|---|---|---|---|
| Raw material | formula | Supplier | no. | Amount | |
| Zinc Chloride | ZnCl₂ | Merck 108816 | 102-4965 | 6.8 | g |
| Copper Sulfate | CuSO₄•5H₂O | Merck 102790 | 109-0771 | 2.5 | g |
| Nickel Chloride anhydrous | NiCl2 | Merck 806722 | 101-6652 | 0.13 | g |
| Iron Sulfate | FeSO4•7H₂O | Merck 103965 | | 13.9 | g |
| Manganese Sulfate | MnSO₄•H₂O | Merck 105941 | | 8.45 | g |
| Citric acid | C₆H₈O₇•H₂O | Merck 100244 | | 3 | g |
| Ion exchanged water up | | | | 1000 | ml |
| to | | | | | |

COVE sucrose plates were composed of 342 g of sucrose, 20 g of agar powder, 20 ml of COVE salt solution, and deionized water to 1 liter. The medium was sterilized by autoclaving at 15 psi for 15 minutes (Bacteriological Analytical Manual, 8th Edition, Revision A, 1998). The medium was cooled to 60° C. and 10 mM acetamide, Triton X-100 (50 μl/500 ml) were added. COVE salt solution was composed of 26 g of MgSO₄.7H₂O, 26 g of KCL, 26 g of KH₂PO₄, 50 ml of COVE trace metal solution, and deionized water to 1 liter.

COVE trace metal solution was composed of 0.04 g of Na₂B₄O₇·10H₂O, 0.4 g of CuSO₄·5H₂O, 1.2 g of FeSO₄·7H₂O, 0.7 g of MnSO₄·H₂O, 0.8 g of Na₂MoO₄·2H₂O, 10 g of ZnSO₄·7H₂O, and deionized water to 1 liter.

### Example 5: Rasamsonia Eburnean PLC; Cloning and Expression

The phospholipase encoding gene was cloned by conventional techniques from the strain indicated and inserted into plasmid pCaHj505 (WO 2013/029496 Example 1: Cloning and expression).

The phospholipase encoding gene was cloned by conventional techniques from the strain indicated and inserted into plasmid pCaHj505 (WO 2013/029496). The gene was expressed with the native secretion signal having the following amino acid sequence MRAFLITALASLATAAGA (amino acid residues 1 to 18 of SEQ ID NO: 22).

Expression in A. Oryzae

One clone with the correct recombinant gene sequence was selected and the corresponding plasmid was integrated into the Aspergillus oryzae MT3568 host cell genome. A. oryzae MT3568 is an amdS (acetamidase) disrupted gene derivative of A. oryzae JaL355 (WO 02/40694) in which pyrG auxotrophy was restored by disrupting the A. oryzae acetamidase (amdS) gene with the pyrG gene.

The hydrolytic activity of the phospholipase produced by the Aspergillus transformants was investigated using lecithin/agarose plates (plate assay described in assay section). 20 μl aliquots of the culture broth from the different transformants, or buffer (negative control) were distributed into punched holes with a diameter of 3 mm and incubated for 1 hour at 37° C. The plates were subsequently examined for the presence or absence of a dark violet zone around the holes corresponding to phospholipase activity.

A recombinant Aspergillus oryzae clone containing the integrated expression construct was selected and it was cultivated in 2400 ml of YPM medium (10 g yeast extract, 20 g Bacto-peptone, 20 g maltose, and deionised water to 1000 ml) in shake flasks during 3 days at a temperature of 30° C. under 80 rpm agitation. Culture broth was harvested by filtration using a 0.2 μm filter device. The filtered fermentation broth was used for enzyme characterization. The gene was expressed with the native secretion signal having the following amino acid sequence MRAFLITALASLATAAGA (amino acid residues 1 to 18 of SEQ ID NO: 22).

Purification

The culture supernatant was firstly precipitated by (NH₄)₂SO₄, and then dialyzed with 20 mm Bis-Tris at pH 6.5. Then the sample was applied to chromatographic column of Q Sepharose Fast Flow (GE Healthcare) equilibrated with 20 mm Bis-Tris at pH 6.5. A gradient increase of NaCl concentration was applied from zero to 0.35M NaCl with 15 CV (column volume), then to 0.5M NaCl with 3 CV, finally to 1M NaCl with 2 CV. The fractions and samples pass the column (flowthrough fraction) were checked by SDS-PAGE. Based on SDS figure, fractions from No. 18 to No. 43 were collected and added (NH₄)₂SO₄ to a final concentration of 1.2M.

The pooled fractions were loaded into a column of Phenyl Sepharose 6 Fast Flow (GE Healthcare) equilibrated with 20 mm Bis-Tris at pH 6.5 with 1.2M (NH₄)₂SO₄ added. A gradient decrease of (NH₄)₂SO₄ concentration was applied from 1.2M to Zero. The elution fractions and flowthrough fraction were collected and tested for PLC activity by Lecithin plate at pH5.5. The fractions with PLC activity were checked by SDS-PAGE. Both elution fractions from No. 1 to No. 18 and flowthrough fraction had good PLC activity and purity, were picked up as target proteins.

N- and C-Terminal Processing

N-Terminal Sequencing:

N-terminal sequencing analyses were performed using an Applied Biosystems Procise® protein sequencing system. The samples were purified on a Novex® precast 4-20% SDS polyacrylamide gel (Life Technologies). The gel was run according to manufacturer's instructions and blotted to a ProBlott® PVDF membrane (Applied Biosystems). For N-terminal amino acid sequencing the main protein band was cut out and placed in the blotting cartridge of the Procise® protein sequencing system. The N-terminal sequencing was carried out using the method run file for PVDF membrane samples (Pulsed liquid PVDF) according to manufacturer's instructions. The N-terminal amino acid sequence was deduced from the 7 chromatograms corresponding to amino acid residues 1 to 7 by comparing the retention time of the peaks in the chromatograms to the retention times of the PTH-amino-acids in the standard chromatogram.

Protein Identification by Mass Spectrometry (MS/MS) Sequencing:

Protein identification was performed by tandem mass spectrometry (MS/MS) analysis of tryptic peptides from an in gel digest. First the sample was reduced by DTT and alkylated with lodacetamide. The reduced and alkylated sample was then applied to SDS-gel electrophoresis.

The gel was run and stained according to manufacturer's instructions (Novex® precast 4-20% SDS polyacrylamide gel (Life Technologies). The main protein band was cut out and the gel piece digested over night by Sequencing Grade trypsin (Roche). Following digestion the generated tryptic peptides were extracted and analysed on an Orbitrap LTQ XL mass spectrometer (Thermo Scientific) where peptide masses and peptide fragment masses are measured. For protein identification the experimentally obtained masses were compared with the theoretical peptide masses and peptide fragment masses of proteins stored in databases by the mass search program Mascot (Matrix science).

Determination of Molecular Weight:

The intact molecular weight analyses were performed using a MAXIS II electrospray mass spectrometer (Bruker Daltonik GmbH, Bremen, DE). The samples were diluted to 1 mg/ml in MQ water. The diluted samples were applied to an Aeris Widepore C4 column (Phenomenex). The samples were washed and eluted from the column running an acetonitrile linear gradient and introduced to the electrospray source with a flow of 300 ml/min by an Ultimate 3000 LC system (Dionex). Data analysis is performed with DataAnalysis version 4.2 (Bruker Daltonik GmbH, Bremen, DE). The molecular weight of the samples was calculated by deconvolution of the raw data in the range 20.000 to 80.000 Da.

Substrate Specificity

P-NMR Assay of Purified PLC Enzymes

Concept

The assay was conducted by incubating the PLC with a 10:1 mixture of a crude vegetable oil and aqueous citrate buffer pH 5.5. Enzyme concentration was 30 mg/kg (mg EP per kg oil). The mixture was incubated with vigorous shaking at 50 C for 2 h. The reaction mixture was then analyzed by 31P NMR. This involves an aqueous extraction step during which the phosphor species liberated by the PLC are removed from the oil phase. Hence, only lipophilic P-species are detected, i.e. unreacted phospholipid.

Enzymes:

Mature polypeptide of SEQ ID NO: 22 (Rasamsonia PLC), SEQ ID NO: 25 (T. Spiralis PLC) and SEQ ID NO: 28 (T. harzianum PLC),

Assay Procedure

The purified enzyme was diluted to 0.27 mg/mL in 100 mM citrate buffer pH 5.5. The assay was initiated by adding 25 uL diluted enzyme to 250 uL crude vegetable oil in a 2 mL Eppendorf tube and incubating the mixture in a thermoshaker at 50 C for 2 h. The oil used was a crude soybean oil containing a significant amount of both PA, PE, PI and PC (100-200 ppm P of each).

NMR Analysis

To the oil sample was then added 0.500 mL internal standard (IS) solution, followed by 0.5 mL CDCl₃ and 0.5 mL Cs-EDTA buffer. The sample was shaked for 5 min, and then centrifuged (tabletop centrifuge, 5 min, 13,400 rpm) to get phase separation. The lower phase was transferred to a NMR-tube. P-NMR was performed with 128 scans and a delay time of 5 s. All signals were integrated. Assignments (approx. ppm): 1.5 (PA), −0.1 (PE), −0.6 (PI), −0.8 (PC). The concentration of each species was calculated as “ppm P”, i.e. mg elemental P per kg oil sample. Hence, ppm P=I/I(IS)*n(IS)*M(P)/m(oil). Residual phospholipid content was calculated as the ratio of enzyme treated sample vs blank. The internal standard solution is 2 mg/mL triphenylphosphate in methanol. The Cs-EDTA buffer was prepared as: EDTA (5.85 g) is dispersed in water (approx. 50 mL). The pH was adjusted to 7.5 using 50% w/w CsOH. This gave a clear solution. Water was added up to 100 mL to give a concentration of 0.2 M EDTA.

Results

Table 2 below shows residual phospholipid content in percent (0 is full hydrolysis, 100 is no hydrolysis).

TABLE 2

| | | | | |
|---|---|---|---|---|
| Batch# | PA | PE | PI | PC |

Spiralis

| | | | | |
|---|---|---|---|---|
| U4G2D | 53 | 78 | 41 | 35 |
| U3CC8 | 30 | 34 | 0 | 10 |
| U3EY3 | 44 | 60 | 31 | 17 |
| U3CWK | 60 | 75 | 37 | 39 |
| U3CWJ | 52 | 69 | 42 | 32 |
| U3CWH | 59 | 72 | 38 | 35 |
| U3CC7 | 54 | 72 | 38 | 33 |

Rasamsonia

| | | | | |
|---|---|---|---|---|
| U4BCJ | 53 | 72 | 37 | 14 |

Harzianum

| | | | | |
|---|---|---|---|---|
| U4AVE | 21 | 44 | 7 | 11 |
| U4AVF | 75 | 85 | 48 | 46 |
| U4AVG | 26 | 43 | 7 | 6 |
| U39PX | 46 | 67 | 36 | 29 |
| U49A3 | 25 | 55 | 21 | 17 |

DCS-Temp Profile

Determination of Td by Differential Scanning calorimetry.

The thermostability of Harzianum (U49A3) was determined by Differential Scanning calorimetry (DSC) using a VP-Capillary Differential Scanning calorimeter (MicroCal Inc., Piscataway, NJ, USA). The thermal denaturation temperature, Td (° C.), was taken as the top of denaturation peak (major endothermic peak) in thermograms (Cp vs. T) obtained after heating enzyme solutions (approx. 0.5 mg/ml) in buffer (50 mM acetate buffer pH 5.0) at a constant programmed heating rate of 200 K/hr.

Sample- and reference-solutions (approx. 0.2 ml) were loaded into the calorimeter (reference: buffer without enzyme) from storage conditions at 10 deg C. and thermally pre-equilibrated for 20 minutes at 20° C. prior to DSC scan from 20° C. to 100° C. Denaturation temperatures were determined at an accuracy of approximately +/−1° C. Td obtained under these conditions for U49A3 was 79 deg C.

The thermostability of Spiralis (U4G2D) was determined by Differential Scanning calorimetry (DSC) using a VP-Capillary Differential Scanning calorimeter (MicroCal Inc., Piscataway, NJ, USA). The thermal denaturation temperature, Td (° C.), was taken as the top of denaturation peak (major endothermic peak) in thermograms (Cp vs. T) obtained after heating enzyme solutions (approx. 0.5 mg/ml) in buffer (50 mM acetate buffer pH 5.5) at a constant programmed heating rate of 200 K/hr.

Sample- and reference-solutions (approx. 0.2 ml) were loaded into the calorimeter (reference: buffer without enzyme) from storage conditions at 10 deg C. and thermally pre-equilibrated for 20 minutes at 20° C. prior to DSC scan from 20° C. to 100° C. Denaturation temperatures were determined at an accuracy of approximately +/−1° C. Td obtained under these conditions for U4G2D was 67 deg C.

The thermostability of Rasamsonia (U4BCJ) was determined by Differential Scanning calorimetry (DSC) using a VP-Capillary Differential Scanning calorimeter (MicroCal Inc., Piscataway, NJ, USA). The thermal denaturation temperature, Td (° C.), was taken as the top of denaturation peak (major endothermic peak) in thermograms (Cp vs. T) obtained after heating enzyme solutions (approx. 0.5 mg/ml) in buffer (50 mM acetate buffer pH 5.5) at a constant programmed heating rate of 200 K/hr.

Sample- and reference-solutions (approx. 0.2 ml) were loaded into the calorimeter (reference: buffer without enzyme) from storage conditions at 10 deg C. and thermally pre-equilibrated for 20 minutes at 20° C. prior to DSC scan from 20° C. to 100° C. Denaturation temperatures were determined at an accuracy of approximately +/−1° C. Td obtained under these conditions for U4BCJ was 82 deg C.

Degumming Performance

Performance of the phospholipase C enzyme of the present invention Rasamsonia, Harzianum and Spiralis were tested in a degumming assay that mimics industrial scale degumming. The assay measured one of or both of the following parameters in the oil phase after the degumming procedure described in degumming assay paragraph below.
a) Diglyceride content by High-performance liquid chromatography (HPLC) coupled to Charged Aerosol Detector (Corona Veo) according to the principles described in AOCS Official Method Cd 11d-96. DIONEX equipment and Lichrocart Si-60, 5 μm, Lichrosphere 250-4 mm, MERCK column was applied.
b) Total phosphorus and other metals such as Ca, Mg, Zn measured by Inductively coupled plasma optical emission spectrometry (ICP-OES) with an accuracy of approximately ±1 ppm P.

Degumming Assay

Crude soybean oil (75 g) was initially acid/base pretreated to facilitate conversion of insoluble phospholipids salt into more hydratable forms and ensure an environment suitable for the enzyme. Acid/base pretreatment was done by acid addition of either Ortho Phosphoric acid (75% solution) or citric acid (50% solution). Acid was applied in amounts equal to either 0.065% or 0.09% (100% pure Ortho Phosphoric acid/100% pure citric acid) based on oil amount and mixing in ultrasonic bath (BRANSON 5800) for 5 min and incubation in rotator for 15 min. This was followed by base neutralization with 1 M NaOH applied in equivalents (from 0.45 to 5) to pure Ortho Phosphoric acid (i.e., the acid which was used in pretreatment) and mixed in ultrasonic bath for 5 min. The enzyme reaction was conducted in low aqueous system (3% water total based on oil amount) in 100 ml centrifuge tubes, cylindrical, conical bottom. Samples were ultrasonic treated for 5 min, followed by incubation in a heated cabinet at selected temperature (from 60 to 70° C.) with stirring at 20 rpm for a selected incubation time (from 1 to 24 hours). To separate the mixture into an oil phase and a heavy water/gum phase the samples were centrifuged at 700 g at 85° C. for 5 min (Koehler Instruments, K600X2 oil centrifuge).

The phosphorous, calcium, magnesium and zinc composition in the crude soybean oil, used in the experiments, is indicated in Table 3.

TABLE 3
Metal composition of crude oil measured by ICP-OES (mg/kg oil)
Table 3: Metal composition of crude oil measured by ICP-OES (mg/kg oil)

| | P | Ca | Mg | Zn |
|---|---|---|---|---|
| Crude oil 1- FS-2015-00022 | 743 | 168 | 115 | 10 |
| Crude oil 2-ex 2. FS-2015- | 615 | 136 | 85 | 10 |
| 00021 | | | | |
| Crude oil 3 FS-2014-00070. | 631 | 102 | 69 | 3 |
| Crude oil 4-ex 4A FS-2015- | 479 | 146 | 92 | 11 |
| 00021. | | | | |
| Crude oil 5-ex 4B - FS- | 465 | 147 | 93 | 11 |
| 2015-00022 | | | | |
| Crude oil 6-ex 5 FS-2015- | 622 | 157 | 105 | 11 |
| 00023 | | | | |

Examples 6 to 11 below describes results obtained using the degumming assay.

### Example 6: Harzianum U4AVG Compared to Mrs. Marianne U4DB1 at 60° C. (58.6 Identity)

Harzianum (U4AVG) (mature polypeptide of SEQ ID NO: 28) was applied in degumming assay at 60° C. compared against N. mariannaeae (U4DB1) (mature polypeptide of SEQ ID NO: 19) at enzyme dosage of 10 mg enzyme protein per kg oil applying oil 1. The diglyceride content after enzymatic degumming for 2, 5 and 24 hrs were measured (oil pretreated with 0.065% citric acid/1.5 eqv. NaOH) as well as the total phosphorous content after 2, 5 and 24 hours incubation measured by ICP. The results (average of double determination) and standard deviation (STDEV) are presented in table 4A and 4B.

Tables 4A and 48

TABLE 4A
Diglyceride increase (% w/w) after enzyme incubation in oil 1

| | AVE | STDEV | AVE | STDEV | AVE | STDEV |
|---|---|---|---|---|---|---|
| | 2 h | 2 h | 5 h | 5 h | 24 h | 24 h |
| Blank | 0.12 | 0.03 | 0.00 | 0.01 | 0.06 | 0.01 |
| Harzianum | 0.47 | 0.06 | 0.69 | 0.06 | 1.16 | 0.06 |
| Mariannaeae | 0.14 | 0.04 | 0.33 | 0.15 | 0.95 | 0.15 |

TABLE 4B
Total P content after degumming
(mg/kg = ppm) dobb determination

| | AVE | STDEV | AVE | STDEV | AVE | STDEV |
|---|---|---|---|---|---|---|
| | 2 h | 2 h | 5 h | 5 h | 24 h | 24 h |
| Blank | 51 | 1 | 47 | 5 | 43 | 1 |
| Harzianum | 45 | 8 | 34 | 9 | 22 | 6 |
| Mariannaeae | 49 | 3 | 44 | 7 | 23 | 9 |

Degumming with Harzianum (U4AVG) at 60° C. results in superior diglyceride formation compared to diglyceride formation by mariannaeae PLC (U4DB1). Harzianum converts up to 78% of the phospholipids at conditions tested (60° C., 24 hours). Conversion calculation is based on the assumption that 743 ppm P total measured by ICP equals 1.86 wt % phospholipid (Average PL Mw ˜772 g/mol, Mw P˜31 g/mol) equal to max 1.49% DG increase obtainable (80% of phospholipid molecule).

### Example 7: Dose Response Study of Harzianum (U4AVG) at 60° C.

Harzianum (U4AVG) (mature polypeptide of SE ID NO: 28) was applied in degumming assay at 60° C. at various enzyme dosage of 1×-2×-5×-10× mg enzyme protein per kg oil applying oil 2. The diglyceride content after enzymatic degumming for 1, 2 and 4 hrs were measured (oil pretreated with 0.09% phosphoric acid/1.5 eqv. NaOH). The results are presented in table 5.

TABLE 5
Diglyceride increase

| | Table 5: Diglyceride increase (% | | |
|---|---|---|---|
| Enzyme | w/w) after enzyme incubation in oil 2 FS- | | |
| dosage (mg enzyme | 2015-00021 | | |
| protein /kg oil) | 1 hrs | 2 hrs | 4 hrs |
| 1X | 0.05 | 0.21 | 0.23 |
| 2X | 0.06 | 0.30 | 0.38 |
| 5X | 0.32 | 0.42 | 0.41 |
| 10X | 0.59 | 0.87 | 1.06 |

Degumming with Harzianum (U4AVG) at 60° C. showed increased diglyceride formation at increased enzyme dosage in the interval tested (1-10× mg EP/kg oil). Up to 86% of the phospholipids were converted at conditions tested (60° C., 4 hours, 10 mg EP/kg oil). Conversion calculation is based on the assumption that 615 ppm P total measured by ICP is equal to 1.54 wt % phospholipid (Average PL Mw ˜772 g/mol, Mw P˜31 g/mol) equal to max 1.23% DG increase obtainable (80% of phospholipid molecule).

### Example 8: Rasamsonia (U3GPC) Performance Compared to P.Emersonii (U4DB4) and Kionochaeta PLC (U1A3F) at 60° C. Applying Phosphoric Acid for Oil Pretreatment

Rasamsonia (U3GPC) (mature polypeptide of SEQ ID NO: 22) was applied in degumming assay at 60° C. compared against Kionochaeta PLC (U1A3F) (mature polypeptide of (SEQ ID NO: 17) and P.emersonii (U1DW6) (mature polypeptide of SEQ ID NO: 15) at enzyme dosage of 30 mg enzyme protein per kg oil applying oil 3. The diglyceride content after enzymatic degumming for 2, 4, 6 and 24 hrs were measured (oil pretreated with 0.09% phosphoric acid (PA) and +/−1.5 eqv. NaOH) as well as the total phosphorous content after 2 and 24 hours incubation measured by ICP. The results are presented in table 6.

TABLE 6
Diglyceride increase (% w/w)
Table 6: Diglyceride increase (% w/w) and end P content
after enzyme incubation in oil 3 FS-2014-00070

| | | | | | | Total P by ICP | |
|---|---|---|---|---|---|---|---|
| | | DG increase (wt %) as function | | | | after degumming | |
| | Oil Pre- | of reaction time (hours) | | | | of x hours | |
| Enzyme | treatment | 2 | 4 | 6 | 24 | 2 | 24 |
| Blank | 0.09% PA + 1.5 | 0.08 | 0.07 | 0.06 | 0.14 | 34 | 32 |
| | eqv | | | | | | |
| Rasamsonia | 0.09% PA + 1.5 | 0.45 | 0.50 | 0.59 | 0.83 | 47 | 32 |
| PLC | eqv | | | | | | |
| Kionochaeta | 0.09% PA + 1.5 | 0.25 | 0.28 | 0.40 | 0.84 | 41 | 24 |
| PLC | eqv | | | | | | |
| Emersonii | 0.09% PA | 0.40 | 0.56 | 0.66 | 0.76 | 22 | 56 |

Degumming with Rasamsonia at 60° C. showed accelerated diglyceride formation compared to Kion PLC during first 6 hours (applying same oil preateatment) and almost identical performance to P. emersonii tested without any caustic (NAOH) addition. Rasamsonia resulted in conversion of up to 66% of the phospholipids at conditions tested (60° C., 24 hours, 30 mg EP/kg oil, 0.09% phosphoric acid+1.5 eqv. NaOH). Conversion calculation is based on the assumption that 631 ppm P total measured by ICP is equal to 1.58 wt % phospholipid (Average PL Mw ˜772 g/mol, Mw P˜31 g/mol) equal to max 1.26% DG increase obtainable (80% of phospholipid molecule).

### Example 9: Rasamsonia (U4BCJ) Performance at 60° C. and 70° C. with and without Oil Pretreatment Applying Citric Acid for Oil Pretreatment (70C) and_(60C)

Rasamsonia (U4BCJ) (mature polypeptide of SEQ ID NO: 22) was applied in degumming assay at 60° C. and 70° C. at enzyme dosage of 10 mg enzyme protein per kg oil applying oil 4/oil 5, and compared with P. emersonii PLC (mature polypeptide of SEQ ID NO: 15). The diglyceride content after enzymatic degumming for 2, 5 and 24 hrs were measured. The results are presented in table 7A and 7B.

Tables 7A and 78

TABLE 7A
Diglyceride increase (% w/w) after enzyme incubation
at 70° C. in oil 4. FS-2015-00021.

| | | Reaction time (hours) | | |
|---|---|---|---|---|
| Enzyme | Oil Pre-treatment | 2 | | 24 |
| none | None | 0.02 | .00 | 0.14 |
| Rasamsonia | None | 0.20 | .32 | 0.74 |
| none | 650 ppm CA + 0.4 eqv NaOH | 0.01 | .00 | 0.00 |
| Rasamsonia | 650 ppm CA + 0.4 eqv NaOH | 0.10 | .13 | 0.92 |

TABLE 7B
Diglyceride increase (% w/w) after enzyme incubation
at 60° C. in oil 5. FS-2015-00022.

| | | Reaction time (hours) | | |
|---|---|---|---|---|
| | Oil Pre-treatment | 2 | 5 | 24 |
| Blank | Water degumming | 0.05 | 0.00 | 0.03 |
| Rasamsonia | Water degumming | 0.23 | 0.48 | 0.99 |
| Blank | 650 ppm CA + 0.4 eqv | 0.04 | 0.06 | 0.00 |
| | NaOH | | | |
| Rasamsonia | 650 ppm CA + 0.4 eqv | 0.00 | 0.01 | 0.46 |
| | NaOH | | | |
| P. emersonii | 650 ppm CA + 0.4 eqv | 0.15 | 0.23 | 0.69 |
| | NaOH | | | |
| Blank | 650 ppm CA + 1.0 eqv | 0.02 | 0.00 | 0.03 |
| | NaOH | | | |
| Rasamsonia | 650 ppm CA + 1.0 eqv | 0.05 | 0.11 | 0.55 |
| | NaOH | | | |
| P. emersonii | 650 ppm CA + 1.0 eqv | 0.01 | 0.08 | 0.90 |
| | NaOH | | | |

Degumming with Rasamsonia showed increased diglyceride formation over time and good performance at 60° C. as well as 70° C. in oil pretreated with citric acid and caustic as well as without any pretreatment of the oil. Full conversion ˜96-100% of the phospholipids was obtained at 70° C., 24 hours, 10 mg EP/kg oil, 650 ppm CA+0.4 eqv NaOH as well as 60° C., 24 hours, 10 mg EP/kg oil, no oil pre-treatment). Conversion calculation is based on the assumption that 465-479 ppm P total measured by ICP is equal to ˜1.2 wt % phospholipid (Average PL Mw ˜772 g/mol, Mw P˜31 g/mol) equal to max ˜0.96% DG increase obtainable (80% of phospholipid molecule).

### Example 10: Spiralis (U4G2D) Performance Compared to Marianneaea (U4DB1) at 60° C.

Spiralis (U4G2D) (mature polypeptide of SEQ ID NO: 25) was applied in degumming assay at 60° C. compared against Mariannaeae (U4DB1) (mature polypeptide of SEQ ID NO: 19 at enzyme dosage of 10 mg enzyme protein per kg oil applying crude oil 5. The diglyceride content after enzymatic degumming for 2, 5 and 24 hrs were measured (oil pretreated with 0.065 citric acid and 1.5 eqv. NaOH) as well as the total phosphorous content after 5 and 24 hours incubation measured by ICP. The results (average of double determination) are presented in table 8.

TABLE 8
Table 8: Diglyceride increase (% w/w) and end P content after
enzyme incubation at 60° C. in oil 5. FS-2015-00023.

| | | | | Total P by ICP after | |
|---|---|---|---|---|---|
| | | | | degumming of x | |
| | Reaction time (hours) | | | hours | |
| | 2 | 5 | 24 | 5 | 24 |
| Blank | 0.05 | 0.06 | 0.12 | 87 | 76 |
| Spiralis | 0.23 | 0.34 | 0.66 | 32 | 28 |
| Mariannaeae | 0.15 | 0.26 | 0.81 | 35 | 24 |

Spiralis performed well under reaction conditions tested and showed faster diglyceride formation after 2 and 5 hours compared to Mariannaeae which showed highest DG formation after 24 h. Spiralis resulted in up to 53% conversion of the phospholipids at conditions tested (60° C., 24 hours, 10 mg EP/kg oil, 0.065% phosphoric acid+1.5 eqv. NaOH). Conversion calculation is based on the assumption that 622 ppm P total measured by ICP is equal to 1.56 wt % phospholipid (Average PL Mw ˜772 g/mol, Mw P˜31 g/mol) equal to max 1.24% DG increase obtainable (80% of phospholipid molecule).

### Example 11: Harzianum, Rasamsonia and Spiralis PLC's Performance Comparison to Mariannaeae, Kionochaeta sp. PLC and P. emersonii PLC at 60° C.

Degumming

| | |
|---|---|
| Harzianum | U4AVE |
| Rasamsonia | U4BCJ |
| Spiralis | U4G2D |
| Kiono in A. | U4GD4 |
| niger | |
| Kiono in A. | U75FP |
| oryzae | |
| Mariannaeae | U4DB1 |
| Emersonii | U4DB4 |

Harzianum, Rasamsonia and Spiralis (mature polypeptides of SEQ ID NOs: 28, 22 and 25, respectively) were applied in degumming assay at 60° C. compared against Kionochaeta sp. PLC (mature polypeptide of SEQ ID NO: 17) expressed either in A. niger or in A. oryzae, Mariannaeae and P. emersonii PLC (mature polypeptide of SEQ ID NO: 19 and 15, respectively) at enzyme dosage of 10 mg enzyme protein per kg oil applying crude oil 8. The oil was pre-treated with 0.065% citric acid and 0.4 molar equivalents or 1.5 molar equivalents NaOH before degumming with P. emersonii PLC and all other enzymes, respectively. The diglyceride contents after enzymatic degumming for 2, 5 and 24 hrs were measured by HPLC, and the total phosphorous content after 5 and 24 hours incubation measured by ICP. The results are presented in table 9.

TABLE 9
Table 9: Diglyceride increase (% w/w) and end P content after
enzyme incubation at 60° C. in oil 7. FS-2015-00025.

| | DG increase after | | | Total P by ICP after | |
|---|---|---|---|---|---|
| | degumming of x | | | degumming of x | |
| | hours (% w/w) | | | hours (ppm) | |
| | Reaction time (hours) | | | | |
| | 2 | 5 | 24 | 5 | 24 |
| Blank | 0.12 | 0.04 | 0.10 | 57 | 61 |
| Harzianum | 0.65 | 0.84 | 1.12 | 16 | 20 |
| Rasamsonia | 0.26 | 0.17 | 0.39 | 72 | 56 |
| Spiralis | 0.20 | 0.22 | 0.46 | 65 | 50 |
| Kiono in A. niger | 0.21 | 0.36 | 0.89 | 50 | 48 |
| Kiono in A. oryzae | 0.29 | 0.51 | 1.04 | 43 | 24 |
| Mariannaeae | 0.24 | 0.38 | 0.97 | 51 | 45 |
| Emersonii | 0.19 | 0.43 | 0.86 | 31 | 18 |

Under the given reaction conditions (60° C., 10 mg EP/kg oil, 0.065% citric acid+1.5 eqv. NaOH) degumming with Harzianum PLC resulted in faster diglyceride increase and phosphorus reduction compared to the other PLC enzymes. Also the highest diglyceride content (1.12% w/w) after 24 h was reached by Harzianum PLC, corresponding to approx. 97% conversion of the phospholipids. Conversion calculation is based on the assumption that 574 ppm P total measured by ICP is equal to 1.44 wt % phospholipid (Average PL Mw ˜772 g/mol, Mw P˜31 g/mol) equal to max 1.15% DG increase obtainable (80% of phospholipid molecule).

Quantitative Analysis of Phospholipids by LCMS/MS

Liquid Chromatography coupled to triple quadrupole mass spectrometer (LC/MS/MS) or coupled to quadrupole mass spectrometer time of flight (LC/TOF/MS) was used to quantify the individual phospholipids species: phosphatidylcholine (PC); Phosphatidylinositol (PI); Phosphatidylethanolamine (PE) and Phosphatidic acid (phosphatidate) (PA). The sensitivity of the assay goes down to less than 1 mg Phosphorus/kg oil for PC, PE and PI (ppm) and less than 10 mg Phosphorus/kg for PA. The oil sample was dissolved in chloroform. The extract was then analysed on LC-TOF-MS (or on LC-MS/MS if lower detection limits are needed) using following settings:

LC-Settings
Eluent A: 50% Acetonitril, 50% Water, 0.15% formic acid
Eluent B: 100% Isopropionic acid, 0.15% formic acid
Run time: 26.9 min
Flow: 0.50 mL/min
Column temperature: 50° C.
Autosampler temp: 15-25° C.
Injection volume: 1 μL
Column type Material: Charged Surface Hybrid, length: 50 mm, size: 1.7 μm, ID: 2.1 mm

MS-Settings

| TOF/MS | MS/MS (Xevo) |
|---|---|
| Capillary: 3.50 kV | Capillary: +3.50/−2.0 kV |
| Cone: 28 | Cone: Component specific |
| Extractor: 2 V | Extractor: 2.5 V |
| RF-lens: 0.5 V | RF-lens: |
| Source temp: 125° C | Source temp: 150° C. |
| Desolvation temp: 500° C. | Desolvation temp: 500° C. |
| Cone gas flow: 30 L/hour | Cone gas flow: 30 L/hour |
| Desolvation gas flow: 850 L/hour | Desolvation gas flow: 850 L/hour |

The data was processed using MassLynx version 4.1 Software. In the below examples the method is just termed LCMS.

TABLE 10
Results
Table 10: Phospholipid content [ppm P] in oil after enzyme
incubation at 60° C. in oil 8, determined by LC-MS

| | Reaction time | | | | | | | | | |
|---|---|---|---|---|---|---|---|---|---|---|
| Enzyme | (hours) | LysoPA | LysoPC | LysoPE | LysoPI | PA | PC | PE | PI | Sum |
| Blank | 2 | 0.3 | 0.0 | 0.0 | 0.0 | 55.5 | 3.2 | 11.6 | 2.4 | 73.2 |
| Harzianum | 2 | 0.0 | 0.0 | 0.0 | 0.0 | 15.3 | 2.5 | 8.4 | 3.8 | 30.2 |
| Rasamsonia | 2 | 0.4 | 0.0 | 0.0 | 0.0 | 58.8 | 3.6 | 10.9 | 2.0 | 75.7 |
| Spiralis | 2 | 0.2 | 0.0 | 0.0 | 0.0 | 48.8 | 4.5 | 13.9 | 2.5 | 69.9 |
| Kiono in A. | 2 | 0.6 | 0.0 | 0.0 | 0.0 | 43.9 | 3.5 | 13.2 | 2.8 | 64.0 |
| niger | | | | | | | | | | |
| Kiono in A. | 2 | 0.5 | 0.0 | 0.0 | 0.0 | 34.1 | 3.1 | 10.2 | 3.3 | 51.3 |
| oryzae | | | | | | | | | | |
| Mariannaeae | 2 | 0.3 | 0.0 | 0.0 | 0.0 | 37.2 | 3.9 | 10.9 | 2.3 | 54.5 |
| Emersonii | 2 | 0.1 | 0.0 | 0.0 | 0.0 | 13.5 | 4.9 | 12.3 | 4.7 | 35.7 |
| Blank | 5 | 0.3 | 0.0 | 0.0 | 0.0 | 47.7 | 3.6 | 14.0 | 2.8 | 68.5 |
| Harzianum | 5 | 0.0 | 0.0 | 0.0 | 0.0 | 3.4 | 2.2 | 4.2 | 2.7 | 12.4 |
| Rasamsonia | 5 | 0.4 | 0.0 | 0.1 | 0.1 | 47.4 | 6.1 | 13.8 | 7.0 | 74.8 |
| Spiralis | 5 | 0.3 | 0.0 | 0.0 | 0.0 | 32.0 | 3.5 | 11.7 | 3.3 | 50.8 |
| Kiono in A. | 5 | 0.3 | 0.0 | 0.0 | 0.0 | 25.6 | 4.1 | 8.4 | 2.4 | 40.8 |
| niger | | | | | | | | | | |
| Kiono in A. | 5 | 0.6 | 0.0 | 0.0 | 0.0 | 19.2 | 2.8 | 8.3 | 2.6 | 33.7 |
| oryzae | | | | | | | | | | |
| Mariannaeae | 5 | 0.2 | 0.0 | 0.0 | 0.0 | 18.4 | 3.3 | 10.5 | 2.3 | 34.7 |
| Emersonii | 5 | 0.0 | 0.0 | 0.0 | 0.0 | 9.1 | 3.9 | 11.0 | 3.4 | 27.5 |
| Blank | 24 | 0.3 | 0.0 | 0.1 | 0.0 | 36.8 | 6.5 | 14.7 | 5.1 | 63.6 |
| Harzianum | 24 | 0.0 | 0.0 | 0.0 | 0.0 | 0.9 | 0.1 | 0.8 | 0.7 | 2.4 |
| Rasamsonia | 24 | 0.4 | 0.0 | 0.1 | 0.0 | 29.3 | 2.6 | 14.4 | 4.8 | 51.6 |
| Spiralis | 24 | 0.2 | 0.0 | 0.0 | 0.0 | 24.0 | 2.5 | 14.9 | 2.4 | 44.1 |
| Kiono in A. | 24 | 0.1 | 0.0 | 0.0 | 0.0 | 8.0 | 1.7 | 8.6 | 2.4 | 20.9 |
| niger | | | | | | | | | | |
| Kiono in A. | 24 | 0.0 | 0.0 | 0.0 | 0.0 | 1.2 | 0.8 | 2.5 | 1.6 | 6.2 |
| oryzae | | | | | | | | | | |
| Mariannaeae | 24 | 0.2 | 0.0 | 0.0 | 0.0 | 1.4 | 2.5 | 4.4 | 1.9 | 10.4 |
| Emersonii | 24 | 0.2 | 0.0 | 0.0 | 0.0 | 0.6 | 0.5 | 1.7 | 2.0 | 5.1 |

The phospholipid composition of the oils after 2, 5 and 24 h incubation is shown in Table 10. It is seen that the PLC enzymes reduce the content of all four phospholipids upon incubation up to 24 h. Degumming applying Harzianum PLC results in fastest decrease of PA, PE and PC.

### Example 12—NaOH Neutralization Influences Yield

The experiment was performed as described above (see heading Degumming). Specifically, the citric acid was dosed at 650 ppm and the enzyme was dosed at 200 ppm. The enzyme used in all samples was a combination of Bacillus thuringiensis PLC (SEQ ID NO. 11) and Pseudomonas sp. PI specific PLC (SEQ ID NO. 13). The amount of equivalents of NaOH used to neutralize the CA of the pretreatment was varied, see table 11 below. Increasing the NaOH used to 3-5 equivalents of the acid in pre-treatment improves yield and decreases dry matter loss. This is observed in both rapeseed and soybean oil. Thus, securing the right pH in the PLC reaction increases the DG formation.

The samples were as indicated in Table 11 below.

TABLE 11

| | | | Dry matter | Delta DG content |
|---|---|---|---|---|
| Flask | NaOH eqv | Oil type | loss(%)* | (%) at 0.5 hrs |
| 1 | 1.5 | Soya bean oil | 3.5 | 0.49 |
| 2 | 2.0 | Soya bean oil | 3.3 | 0.90 |
| 3 | 3 | Soya bean oil | 2.6 | 1.20 |
| 4 | 3.5 | Soya bean oil | 2.6 | 1.22 |
| 5 | 4 | Soya bean oil | 2.6 | 1.29 |
| 6 | 1.5 | Rapeseed oil | 7.2 | 0.30 |
| 7 | 2.0 | Rapeseed oil | 5.9 | 0.39 |
| 8 | 3.5 | Rapeseed oil | 2.3 | 0.99 |
| 9 | 4.0 | Rapeseed oil | 2.1 | 1.20 |

*dry matter loss and delta DG is measured as described above

Thus, in preferred embodiments, the invention relates to the method according to the invention wherein the NaOH treatment is at least 3.0 eqv to the pre-treatment acid, for example from 3.0 to 6.0, such as 3 to 5,5, 3 to 5.0, 3 to 4.5, or 3 to 4.0 equivalents.

### Example 13—Reducing Residual Phosphor and FFA Content

As shown in Example 12, increasing NaOH can increase the yield as measured by delta DG content, and at the same time reduce dry matter loss.

The degumming was performed in the same manner as described above (see heading Degumming), with the following modifications.

The oil was crude rapeseed oil. The acid pre-treatment was by addition of 750 or 1500 ppm phosphoric acid for 15 mins at 70° C. 1.33, 2.0 or 3.0 equivalents of NaOH to the acid were added to neutralize acid and prepare for enzyme treatment. Enzyme hydrolysis was with Bacillus thuringiensis PLC (SEQ ID NO. 11) and Pseudo-monas sp. PI specific PLC (SEQ ID NO. 13) dosed at 200 ppm, the mixture was 2% water. The mixture was incubated for 2 hrs at 60° C. At the end of hydrolysis, alkaline refining was performed by addition of NaOH, 1707 ppm-2040 ppm using 8% NaOH. The total amount of NaOH in each sample was 2700 ppm, which corresponds to 35% in excess of the FFA in the crude oil (1.3%).

The samples were prepared according to Table 12 below. Results are also given in this table.

TABLE 12
Sample conditions and results

| Treatment/ | | | | | | | | |
|---|---|---|---|---|---|---|---|---|
| Flask ID | 1 | 2 | 3 | 4 | 5 | 6 | 7 | 8 |
| Phosphoric | 750 ppm | | | | 1500 ppm | | | |
| acid in ppm | | | | | | | | |
| 75% | | | | | | | | |
| pH adjustment | 2.0 eqv to acid | | 3.0 eqv to acid | | 1.33 eqv to acid | | 2.0 eqv to acid | |
| 8% NaOH | 25% of all NaOH | | 37% of all NaOH | | 33% of all NaOH | | 49% of all NaOH | |
| Enzyme and | 200 ppm NS40140 and 2% water | | | | | | | |
| water | | | | | | | | |
| Caustic | 2040 ppm NaOH | | 1707 ppm NaOH | | 1820 ppm NaOH | | 1372 ppm NaOH | |
| 8% NaOH | 75% of all NaOH | | 63% of all NaOH | | 67% of all NaOH | | 51% of all NaOH | |
| Total Caustic | 2700 ppm NaOH | | | | | | | |
| Total water | 4.5% | 5.5% | 4.5% | 5.5% | 4.5% | 5.5% | 4.5% | 5.5% |
| Results | | | | | | | | |
| FFA % post | 1.4 | 1.4 | 1.5 | 1.4 | 1.8 | 1.7 | 1.8 | 1.8 |
| acid | | | | | | | | |
| FFA % post | 0.67 | 0.61 | 0.63 | 0.67 | 0.72 | 0.69 | 0.57 | 0.65 |
| enzyme | | | | | | | | |
| Delta DG % | — | (0.29) | (0.33) | (0.31) | (0.19) | (0.45) | 0.99 | 0.97 |
| FFA % in final | 0.05 | 0.04 | 0.04 | 0.04 | 0.09 | 0.12 | ((0.27)) | ((0.29)) |
| sample | | | | | | | | |
| Phosphor | ((14) | ((16)) | ((23)) | ((22)) | 1.5 | 3.6 | 5.7 | 5.2 |
| ppm in final | | | | | | | | |
| sample | | | | | | | | |
| Yield | 96.4% | 95.6% | 94.7% | 95.2% | 95.7% | 96.1% | 96.9% | 96.6% |
| estimations | | | | | | | | |

* values in double parentheses are Not optimal; values in single parentheses are acceptable though off target; and values with no parentheses are within target.
Phosphor content. Yield estimation, FFA content and delta degumming were determined as described above.

As can be seen from the table 12, in samples where similar amounts of NaOH were added after acid treatment as neutralization (pH adjustment), and in the alkaline refining step (Caustic 8% NaOH), (see Flask ID 7 and 8 in Table 12), the FFA content in the final sample is suboptimal. In contrast, in samples where the alkaline refining step entailed addition of an amount of NaOH corresponding to over 60% of the total NaOH added in the process, the FFA content was acceptable.

The experiments show that the dosage of acid prior to enzyme hydrolysis and after enzyme hydrolysis influence yield.

### Example 14—Enzymatic Degumming—Industrial Scale—Interprocess Acidification

The aim of this experiment was to bring the phosphorous levels in the degummed oil down to less than 30 ppm, preferably below 10 ppm, while at the same time achieving acceptable levels of FFA (i.e, levels from 0.1%-0,2%), using a single separation step.

The following method was performed on a sample of crude oil (180 kg). The oil was heated to 80° C. under gentle agitation of tank (40% of agitator speed). Then a volume corresponding to 650 ppm pure citric acid (CA) was added. CA as a 30% (w/w) solution. The mixture was subjected to high shear mixing for 15 mins using Sylversson HSM (flow through equipment is 1000 Kgs/h), and thereafter to mechanical agitation for 15 mins at 80° C. and at 70% of the agitator speed installed in the reactor. The pH was adjusted then adjusted by addition of 6 mol eqv NaOH. NaOH was added as an 8% (w/w) solution.

The mixture was subjected to high shear mixing for 15 mins using Siversson HSM (flow through equipment is 1000 Kgs/h), and then cooled down to 60° C. 200 ppm of enzyme (Bacillus thuringiensis PLC (SEQ ID NO. 11) and Pseudo-monas sp. PI specific PLC (SEQ ID NO. 13) was added. The enzymatic reaction was allowed to run for 45 mins. The mixture was deactivated by heating up the oil to 80° C. in the reactor.

Phosphoric acid was added (2.5 kg phosphoric acid/ton oil) and the mixture subjected to high shear mixing for 15 mins. NaOH was added for neutralization using an 8% solution. Nanoneutralization was performed (70 bar) for about 7 minutes, and the oil collected in a tank ready for feeding the GEA centrifuge. This addition of acid after enzymatic hydrolysis, but before alkaline refining step is referred to herein as interprocess acidification.

The above procedure resulted in ca 0.099% FFA and 26 ppm phosphorous in the resulting degummed oil. Thus it can be concluded that the addition of acid post the enzyme stage and prior to the final neutralization stage can ensure full reduction in residual phosphor and FFA on oils where the amount of alkaline added after the chelation stage causes less efficiency of the post enzyme alkaline neutralization step.

### Example 15—PLC Combinations

Degumming assay was carried out as described above (see heading Degumming assay), with the modification that after the enzyme hydrolysis is performed, a higher amount of alkaline is added.

Various combinations of enzymes were tested, as indicated in the table below.

TABLE 13
Sample condition; pH 4 and 6 at 70° C. for 1 hour for enzymatic reaction and results

| | | | Acid/ | | | | |
|---|---|---|---|---|---|---|---|
| | | | base | | | | |
| | | | conditions | | | | |
| | | | for | Dry | Yield | Delta | |
| | | Enzyme | enzyme | matter | gain | DG | Hydrolyzed |
| Flask | Name | Conc. | reaction | loss (%) | (%) | content | PL (%) |
| 1 | Blank | — | 650 ppm | 5.3 | 0.0 | 0.02 | 23 |
| 2 | Blank | — | citric | | | | 26 |
| 3 | Acid PLA | 30 ppm | acid~pH 4 | 3.8 | 1.5 | 0.02 | 47 |
| 4 | | | | | | | 54 |
| 5 | Bacillus | 200 ppm | 650 ppm | 3.4 | 1.9 | 0.51 | 44 |
| 6 | thuringiensis | | citric | | | | 40 |
| | PLC (SEQ ID | | acid + | | | | |
| | NO. 11) and | | 1.5 eqv. | | | | |
| | Pseudo-monas | | NaOH~pH 6 | | | | |
| | sp. PI specific | | | | | | |
| | PLC (SEQ ID | | | | | | |
| | NO. 13) | | | | | | |
| 7 | Bacillus | 200 ppm | | 2.3 | 3.0 | 1.00 | 68 |
| 8 | macauensis | | | | | | 76 |
| | PLC (SEQ ID | | | | | | |
| | NO: 9) and | | | | | | |
| | Pseudo-monas | | | | | | |
| | sp. PI specific | | | | | | |
| | PLC (SEQ ID | | | | | | |
| | NO. 13) | | | | | | |

We conclude that the use of enzymatic degumming in combination with alkaline refining leads to an increase in the yield gain, and decreased dry matter loss. While all tested combinations of enzymes resulted in an increase, the combination of Bacillus macauensis PLC (SEQ ID NO: 9) and Pseudo-monas sp. PI specific PLC (SEQ ID NO. 13) led to the greatest increase of yield, in combination with the greatest reduction in dry matter loss. This combination also performed best in hydrolysis of phospholipids (as measured after hydrolysis but before alkaline treatment), and in increase of diglyceride content.

Thus the invention in one embodiment relates to the method according to the invention, wherein said phospholipid degrading enzymes comprise at least Pseudomonas sp. PI specific PLC (SEQ ID NO. 13). Preferred embodiments relate to wherein the enzymes comprise or consist of Pseudomonas sp. PI specific PLC (SEQ ID NO. 13) and Bacillus macauensis PLC (mature polypeptide of SEQ ID NO: 9).

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1-62. (canceled)

63. A method for refining a vegetable oil containing phospholipids, comprising subjecting the phospholipids to enzymatic hydrolysis by contacting the vegetable oil with one or more phospholipid degrading enzymes under conditions facilitating hydrolysis of phospholipids thereafter subjecting the vegetable oil to chemical refining.

64. The method of claim 63, wherein the enzymatic hydrolysis of phospholipids is performed in a first reaction vessel and the chemical refining is performed in a second reaction vessel, the two reaction vessels being fluidly connected and/or being connected so as to allow liquid passage from the first to the second reaction vessel.

65. The method of claim 63, wherein the enzymatic hydrolysis of phospholipids is performed in a first reaction vessel and the chemical refining is performed in a second reaction vessel, wherein fluid connection between the reaction vessels or liquid passage from the first to the second reaction vessel is not via a separation device, such as a centrifuge.

66. The method of claim 63, wherein the enzymatic hydrolysis of phospholipids and the chemical refining are performed in the same reaction vessel.

67. The method of claim 64, wherein the chemical refining is performed immediately after the enzymatic hydrolysis; preferably in a continuous process operation.

68. The method of claim 63, wherein
the enzymatic hydrolysis is performed in a reaction mixture comprising a heavy phase and a light phase, and
there is no reduction or no substantial reduction of the heavy phase volume or separation of gums/heavy phase from oil before said chemical refining.

69. The method of claim 63, comprising
(a) Providing a reaction mixture comprising said vegetable oil and the one or more enzymes having phospholipid degrading activity, such as a reaction mixture as defined in claim 2;
(b) Subjecting the reaction mixture to conditions allowing enzymatic hydrolysis of phospholipids in the oil, to provide a reacted mixture of said vegetable oil; and
(c) Subjecting the reacted mixture of said vegetable oil to chemical refining.

70. The method of claim 63, further comprising a step of acidification, which is performed after enzymatic hydrolysis and prior to chemical refining.

71. The method of claim 63, comprising subjecting the vegetable oil to water degumming before contacting it with the one or more phospholipid degrading enzymes.

72. The method of claim 63, wherein the vegetable oil is selected from the group consisting of acai oil, almond oil, babassu oil, blackcurrant seed oil, borage seed oil, canola oil, cashew oil, castor oil, coconut oil, coriander oil, corn oil, cottonseed oil, crambe oil, flax seed oil, grape seed oil, hazelnut oil, hempseed oil, jatropha oil, jojoba oil, linseed oil, macadamia nut oil, mango kernel oil, meadowfoam oil, mustard oil, neat's foot oil, olive oil, palm oil, palm kernel oil, palm olein, peanut oil/ground nut oil, pecan oil, pine nut oil, pistachio oil, poppy seed oil, rapeseed oil, rice bran oil, safflower oil, sasanqua oil, sesame oil, shea butter, soybean oil, sunflower seed oil, tall oil, tsubaki oil and walnut oil.

73. The method of claim 63, comprising contacting the vegetable oil with one or more chelation agents capable of complexing Ca and/or Mg ions prior to contacting it with the one or more phospholipid degrading enzymes.

74. An isolated or purified polypeptide having phospholipase A activity, selected from the group consisting of:
(a) A polypeptide having at least 75% sequence identity, such as at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of any one of SEQ ID NOs: 3 and 5,
(b) A polypeptide having at least 75% sequence identity, such as at least 80%, at least 85%, at least 90%, at least 91%, at least 92%, at least 93%, at least 94%, at least 95%, at least 96%, at least 97%, at least 98%, at least 99% or 100% sequence identity to the polypeptide set forth in any one of SEQ ID NOs: 4 and 6;
(c) A fragment of the polypeptide of (a) or (b), that has phospholipase A activity.

75. An isolated or purified polypeptide having phospholipase C activity, selected from the group consisting of:
(a) A polypeptide having at least 60% sequence identity to the mature polypeptide of any one of SEQ ID NOs: 22, 25, 28,
(b) A polypeptide having at least 60% sequence identity to the polypeptide set forth in any one of SEQ ID NOs: 23, 26, 29: and
(c) A fragment of the polypeptide of (a) or (b) that has phospholipase C activity.

76. A composition comprising the polypeptide of claim 74.

77. An isolated or purified polynucleotide encoding the polypeptide of claim 74.

78. A nucleic acid construct or expression vector comprising the polynucleotide of claim 75, wherein the polynucleotide is preferably operably linked to one or more control sequences that direct the production of the polypeptide in an expression host.

79. A recombinant host cell comprising the polynucleotide of claim 77, operably linked to one or more control sequences that direct the production of the polypeptide.

80. A method of producing a polypeptide having phospholipase A activity, comprising cultivating the recombinant host cell of claim 79 under conditions conducive for production of the polypeptide.

81. The method of claim 80, further comprising recovering the polypeptide.
